(19) United States
(12) Patent Application Publication
(10) Pub. No.: US 2011/0112012 A1
(43) Pub. Date: May 12, 2011

(54) Novel Protein for Binding Bacillus Thuringiensis Cry Toxins and Fragments of Cadherins for Enhancing Cry Toxicity Against Dipterans

(75) Inventors: Michael J. Adang, Athens, GA (US); Gang Hua, Athens, GA (US); Mohd Amir Fursan Abdullah, Athens, GA (US); Rui Zhang, Athens, GA (US)

(73) Assignees: The University of Georgia Studies Research Foundation ,Inc., Athens (GA); InsectiGen ,Inc., Athens, GA (US)

(21) Appl. No.: 12/936,027

(22) PCT Filed: Apr. 3, 2009
(86) PCT No.: PCT/US2009/039486

§ 371 Date: Dec. 21, 2010

Related U.S. Application Data
(60) Provisional application No. 61/072,977, filed on Apr. 4, 2008.

(51) Int. Cl. A01N 37/18; A01P 7/04; C07K 14/435; A01H 5/00

(52) U.S. Cl. 514/4.5; 530/324; 800/298

(57) ABSTRACT

The subject invention relates in part to a novel protein for binding Bacillus thuringiensis Cry toxins, and fragments of cadherins for enhancing Cry toxicity against dipterans. The subject invention also relates in part to the discovery that fragments of a midgut cadherin from a dipteran insect synergize Cry proteins that are active against dipterans. Thus, the subject invention includes the use of fragments of cadherin ectodomains for controlling dipterans. Such fragments (that bind Crys) can be administered to a dipteran insect for ingestion. In some preferred embodiments, the source cadherin is a dipteran cadherin. Also in some preferred embodiments, the fragment is administered with a Cry protein that is active against a dipteran. Variants of the fragments of naturally occurring cadherins are included within the scope of the subject invention.

This research was supported by National Institutes of Health Grant R01 AI 29092.

### BACKGROUND

Biopesticides based on the bacterium Bacillus thuringiensis israelensis (Bti) are important tools for controlling mosquitoes and black flies. This is important because mosquitoes vector diseases including malaria, filariasis, dengue, viral encephalitis and West Nile fever. The use of Bti to control the larval stage of mosquitoes provides a critical alternative to chemical agents that mostly control the adult stage. The use as a mosquito larvicide is the primary usage of Bti. Reasons for the increasing usage of biopesticides for mosquito control include emerging incidences of mosquito resistance to chemical pesticides and the environmental consequences of chemical pesticides. Countries and the World Health Organization are encouraging the development and increase of biopesticides for mosquito control. The usage of Bti for mosquito control was recently reviewed (Lacey, L. A. 2007. J. Am. Mosq. Control Assoc. 23: 133-163).

The bacterium Bti is a widely used biopesticide for mosquito control. Bti has been used world-wide for the control of Aedes species that vector dengue fever. There are reports that the use of Bti to control Anopheles mosquitoes reduces malarial incidence. Mosquitoes in the genus Culex, the vector of West Nile Virus, are also controlled by Bti biopesticides.

Bti provides effective control of many species of mosquitoes in different habitats. Factors that affect the efficacy of Bti include rate and amount of ingested Bti, age of larvae (older larvae are more resistant), feeding habits of various mosquito species, settling rate of the Bti, temperature of the water and solar inactivation.

The larvae of all mosquitoes live in water and most species feed on organic matter such as microorganisms and detritus. Some mosquito species, including the anopheline mosquitoes, ingest and gain nutrition from maize pollen (Ye-Ebiyo et al. 2003. Am. J Trop. Med. Hyg. 68: 748-752; Kebede et al. 2005. Am. J. Trop. Med. Hyg. 73: 676-680).

The performance of Bti biopesticides relies on the ingestion of the crystals by mosquito larvae. Therefore, different types of Bti formulations are used to control mosquitoes in different habitats. Common formulations are granular, flowable or even slow-release for control of container breeding mosquitoes. Surface-feeding Anopheles species are best-controlled by formulations that float on the water surface. There has been some development of incorporating Bti crystals into ‘ice granules.’ Recombinant applications of Bti cry genes include engineering into Bacillus thuringiensis, Bacillus sphaericus, E. coli, the protozoan Tetrahymena pyriformis and rice plants. In each case the goal is to control a dipteran insect by producing a Cry toxin in a microorganism that is introduced into the larval habitat where it is ingested. There has also been development of non-viable recombinant organisms that could increase persistence in the environment, such as products based on encapsulated Bt toxins in Pseudomonas fluorescens. This approach ameliorates concerns associated with releasing live genetically engineered microorganisms into the environment.

The specific toxicity of Bti to Anopheles and Aedes and Culex spp. is due to the protein components of the parasporal crystal [reviewed in Federici, et al. 2003. J. Exp. Biol. 206: 3877-85]. The parasporal crystal of Bti is composed of three major insecticidal Cry proteins (Cry4Aa, Cry4Ba, and Cry11Aa) and a cytolytic protein (Cyt1Aa). The Cry4Ba insecticidal protein is highly toxic to Anopheles and Aedes larvae, yet relatively non-toxic to Culex species (Abdullah et al. 2003. Appl. Environ. Microbiol. 69: 5343-53; Delecluse et al. 1993. Appl. Environ. Microbiol. 59: 3922-3927. In contrast, Cry4Aa has low toxicity to Aedes and Culex species, and no toxicity to Anopheles. Bt strains other than Bti produce crystals composed of mosquitocidal Cry proteins. For example, Bt morrisoni produces the same Cry4 and Cry11 proteins as Bti plus an additional Cry protein and Bt jegathesan produces crystals with Cry11Ba. The Cry11Ba protein is more toxic than the related protein, Cry11Aa, to mosquitoes in the three major genera of mosquitoes, Aedes, Anopheles and Culex. Bt jegathesan also produces Cry19Aa, an important protein with high toxicity to Anopheles and Culex larvae.

The Cry4Ba toxin crystal structure has been determined (Boonserm, P. et al. 2005. J. Molec. Biol. 348: 363-82; Puntheeranurak et al. 2005. Ultramicroscopy 105: 115-24). Each domain has a unique role essential to the intoxication process.

Studies on lepidopteran insects revealed several types of Cry toxin receptors: cadherin-like proteins (Vadlamudi, R. K. 1993 J. Biol. Chem. 268: 12334-12340, aminopeptidase N (APN) Knight et al. 1994. Mol. Microbiol. 11: 429-36; Sangadala et al. 1994. J. Biol. Chem. 269: 10088-92); alkaline phosphatase (ALP) (Jurat-Fuentes, J. L. and Adang, M. J. 2004. Eur. J. Biochem. 271: 3127-3135), a glycoconjugate (Valaitis, A. P. et al. 2001. Arch. Insect Biochem. Physiol. 46: 186-200) and glycolipids (Griffitts, J. S. et al. 2005. Science 307: 922-925). An emerging model suggests that these receptor molecules work in a step-wise fashion to mediate toxicity. After binding cadherin, Cry toxin forms a pre-pore oligomer that binds APN and ALP, and inserts into membrane microdomains called lipid rafts (Zhuang, M. et al. 2002. J. Biol. Chem. 277: 13863-72). The insertion of the pre-pore complex into the membrane leads to the formation of ion channels/pores in the brush border membranes of the larval gut leading to cell lysis. Each of these molecules that mediate binding and pore formation has been implicated in resistance development against Cry toxins.

An aminopeptidase N (APN) from Anopheles quadrimaculatus binds Cry11Ba (Abdullah, M. A. et al. BMC Biochem. 7: 16), and an alkaline phosphatase (ALP) from Aedes aegypti (Fernandez, L. E. et al. 2006. Biochem. J. 394: 77-84) was recently identified as a receptor for Cry11Aa. Hua et al. (2008. Biochemistry, In press) identified a cadherin from midgut of An. gambiae (called AgCad1) larvae that functions as a receptor for Cry4Ba toxin.

The use of synergists has been attempted to increase Bt Cry toxicity and to overcome and delay resistance to this biopesticide. Tabashnik et al. (1992. Appl. Environ. Microbiol. 58: 3343-3346) described the phenomenon of synergy for Bt Cry toxins and developed a formula for calculating synergy. Cry proteins are considered synergistic if the combined insecticidal potency is greater than the sum of the individual components. Cry1Aa and Cry1Ac are synergistic in bioassays against gypsy moth larvae (Lee et al. 1996. Appl. Environ. Microbiol. 62: 583-586). Combinations of Bti cytolytic (cyt) toxins with mosquitocidal Cry toxins display synergy in bioassays against mosquito larvae. The Cyt1A toxin of Bti synergizes Cry11A toxicity against yellow fever mosquito Aedes aegypti larvae by functioning as a binding site and insertion into midgut cells (Perez et al. 2005. Proc. Natl. Acad. Sci. U.S.A. 102: 18303-18308). Cyt1A is a cytolysin that is highly toxic not only to mosquito larvae but also to vertebrate and invertebrate cells.

Inhibition of toxicity is accepted evidence for function as a Cry toxin receptor. Typically, a peptide fragment of the receptor (Dorsch, J. A. et al. 2002. Insect Biochem. Mol. Biol. 32: 1025-103636) or a phage mimic of the receptor (Fernandez, L. E. et al. 2006. Biochem. J. 394: 77-84) attenuates Cry in vivo toxicity to larvae. See also Xie, R. et al. 2005. J. Biol. Chem. 280: 8416-8425; Gomez, I. et al. 2001. J. Biol. Chem. 276: 28906-28912. We reported a surprising, opposite effect in which a fragment of Bt-R₁ cadherin, the Cry1A receptor from Manduca sexta, not only bound toxin but enhanced Cry1A toxicity against lepidopteran larvae (Chen, J. et al. 2007. Proc. Natl. Acad. Sci. U.S.A. 104: 13901-13906). If the binding residues within cadherin repeat 12 (CR) were removed, the resulting peptide lost the ability to bind a toxin, Cry1Ab and lost its function as a toxin synergist. See also WO 2005/070214. Cry1Ab binding to the CR12-MPED may promote the switch of toxin from monomer to oligomer according to the Bravo model (Bravo, A. et al. 2004. Biochim Biophys Acta 1667: 38-46).

### BRIEF SUMMARY OF THE INVENTION

The subject invention relates in part to a novel protein for binding Bacillus thuringiensis Cry toxins, and fragments of cadherins for enhancing Cry toxicity against dipterans. The subject invention also relates in part to the discovery that fragments of a midgut cadherin from a dipteran insect synergize Cry proteins that are active against dipterans. Thus, the subject invention includes the use of fragments of cadherin ectodomains for controlling dipterans. Such fragments (that bind Crys) can be administered to a dipteran insect for ingestion. In some preferred embodiments, the source cadherin is a dipteran cadherin. Also in some preferred embodiments, the fragment is administered with a Cry protein that is active against a dipteran. Variants of the fragments of naturally occurring cadherins are included within the scope of the subject invention.

### BRIEF DESCRIPTION OF THE FIGURES

FIG. 1: (A) Diagram of An. gambiae AgCad1 molecule and primer locations. (B) Protein sequence was analyzed using the ISREC ProfileScan server (website hits.isb-sib.ch/Cgi-bin/PFSCAN). Amino acid sequences representing the CR modules are in bold. Amino acids constituting the putative signal leading peptide (LHL . . . to . . . EPR in line 1) and TM (underlined) are in Red; putative calcium binding sites (DRD, DYD, and DPD) are in Green; integrin binding sites (RGD in line 3 and LDV in the line between residues 720 and 800) are in Blue.

FIG. 2: Partially purified AgCAd1 (A) specifically binds Cry4Ba (B, C). The cadherin expressed on S2 cells was solubilized in CHAPS, and then the soluble proteins were loaded on a nickel-chelating Sepharose column and eluted with imidazole. (A) The partially purified proteins were separated on SDS-PAGE and transferred to PVDF filter detected with α-V5 sera. Arrow denotes detected cadherin. (B) The proteins were also dotted on PVDF filter directly and probed with ¹²⁵I-Cry4Ba, or with ¹²⁵I-Cry4Ba plus unlabeled Cry4Ba (1000-fold). (C) The proteins purified from pIZT control and pIZT-AgCad1 cells were dotted on PVDF filter and probed with ¹²⁵I-Cry4Ba, or with ¹²⁵I-Cry4Ba plus unlabeled Cry4Ba or Cry1Ab (1000-fold).

FIG. 3: CR11-MPED peptide enhances Cry4Ba toxicity and displays limited Cry4Ba binding on dot blots. (A) Bioassay of Cry4Ba on A. gambiae with or without truncated cadherin fragments. Fourth instar larvae were put in bioassay wells with 2 ml of distilled water, each well contained 10 larvae. Concentration of Cry4Ba toxin was 0.25 μg/ml with 100-fold of truncated peptides in mass ratio. Control groups contained same amounts of peptides as in test groups. Each column represented the mean±SE from four replicates which were composed of 10×4 A. gambiae larvae. (B) CR11-MPED or TM-Cyto peptides were spotted in duplicate on a membrane filter and probed with ¹²⁵I-Cry4Ba alone or in the presence of 1,000-fold excess of unlabeled Cry4Ba.

FIG. 4: Schematic figure of full length AgCad1 and corresponding partial cadherin fragments (CR9-11 and CR11-MPED) constructs.

FIG. 5: CR9-11 and CR11-MPED AgCad peptides enhance Cry4Ba toxicity to A. aegypti larvae (Panel A). Bioassay of 4^{th} instar A. aegypti larvae using a fixed amount of Cry4Ba protoxin (IBF) (12.5 ng/ml) with increasing ratios of CR9-11 (IBF) or CR11-MPED (IBF). In Panel B, treatments consisted of Cry4Ba alone, Cry4Ba with CR9-11 (IBF) or BtB7 (IBF of CR8-10 of western corn rootworm cadherin) or cadherin fragments alone. Each bioassay consisted of 5 CPB larvae per cup with 10 cups per treatment. Larval mortality was scored 16 h after treatment. Each data point represents data for the mean±standard error from a bioassay, which was conduced with 10 larvae per replicate and four replicates per treatment. Different letters above the standard error bars indicate significant difference between means at α=0.05 (LSD test).

FIG. 6: Dose-toxicity bioassay of A. aegypti 4^{th} instar larvae with fixed mass ratio of 1:25 (Cry4Ba (IBF):cadherin fragment (IBF)) (A) or (Cry4Ba (IBF):cadherin fragment (SF)) (B). Larval mortality was scored 16 h after treatment. The toxicity of Cry4Ba mixtures of either CR11-MPED or CR9-11 was higher than Cry4Ba alone, while the mixture of Cry4Ba+CR9-11 was more toxic compared to the mixture of Cry4Ba+CR11-MPED. Each data point represents data for the mean±standard error from a bioassay, which was conduced with 10 larvae per replicate and four replicates per treatment. Asterisk symbols above the standard error bars indicates significant difference between means at α=0.05 (LSD test) compared between Cry4Ba protoxin (IBF) alone and Cry4Ba protoxin (IBF) with each cadherin fragments.

FIG. 7: Bioassay of 4th instar An. gambiae larvae using fixed amount of Cry4Ba toxin (SF) (0.5 μg/ml) alone or with AgCad CR11-MPED (IBF), AgPCAP CR11-MPED (IBF), or MsCad CR12-MPED (IBF) respectively. Larval mortality was scored 16 h after treatment. Significant increase in mortality was observed with cadherin fragments AgCad and MsCad.

FIG. 8: AgCad fragment binding to Cry4Ba toxin in a microplate binding assay. Microplate wells were coated with Cry4Ba (1 μg/ml) and probed with biotin-AgCad CR9-11 (Panel A) or biotin-AgCad CR11-MPED (B). Non-specific binding was determined by the addition of 1000-fold unlabeled homologous cadherin fragment.

### BRIEF DESCRIPTION OF THE SEQUENCES

SEQ ID NO: 1 is the nucleotide sequence of the full-length AgCad1 molecule.

SEQ ID NO: 2 is the amino acid sequence of the full-length AgCad1 molecule.

SEQ ID NO: 3 is the nucleotide sequence of the CR11-MPED region of the AgCad1 molecule.

SEQ ID NO: 4 is the amino acid sequence of the CR11-MPED region of the AgCad1 molecule.

SEQ ID NO: 5 is the nucleotide sequence of the CR9-11 region of the AgCad1 molecule.

SEQ ID NO: 6 is the amino acid sequence of the CR9-11 region of the AgCad1 molecule.

SEQ ID NO: 7 is the nucleotide sequence of the full-length AgPCAP molecule.

SEQ ID NO: 8 is the amino acid sequence of the full-length AgPCAP molecule.

SEQ ID NO: 9 is the nucleotide sequence of the CR11-MPED region of the AgPCAP molecule.

SEQ ID NO: 10 is the amino acid sequence of the CR11-MPED region of the AgPCAP molecule.

SEQ ID NO: 11 is the nucleotide sequence of the full-length BtR1a molecule.

SEQ ID NO: 12 is the amino acid sequence of the full-length BtR1a molecule.

SEQ ID NO: 13 is the nucleotide sequence of the CR12-MPED region of the BtR1a molecule. The MPED region is nucleotides 298-618.

SEQ ID NO: 14 is the amino acid sequence of the CR12-MPED region of the BtR1a molecule. The MPED region is amino acid residues 100-206. (This polypeptide, and the other relevant polypeptides above, can be used according to the subject invention without the MPED region. The MPED region for the other polypeptides can be determined by sequence alignments.)

### DETAILED DESCRIPTION

The following abbreviations are used herein: alkaline phosphatase (ALP), aminopeptidase N (APN), Bacillus thuringiensis (Bt), Bacillus thuringiensis israelensis (Bti) bovine serum albumin (BSA), cadherin repeat (CR), cytoplasmic (Cyto), 5-(6)-carboxy-tetramethylrhodamine (TAMRA), Drosophila melanogaster S2 cells (S2), isopropyl β-D-thiogalactopyranoside (IPTG), membrane proximal extracellular domain (MPED), polymerase chain reaction (PCR), putative cell adhesion protein (PCAP), rapid amplification of cDNA ends (RACE), and transmembrane (TM).

The subject invention relates in part to the discovery that fragments of insect midgut cadherins synergize Cry proteins that are active against dipterans. Thus, the subject invention relates in part to the use of fragments of insect cadherins, including dipteran cadherin ectodomains, for controlling dipterans. Such fragments (that bind Crys) can be administered to a dipteran insect for ingestion. In some preferred embodiments, the fragment is administered with a Cry protein that is active against a dipteran.

In some embodiments, the subject invention relates to the discovery that An. gambiae cadherin AgCad1 binds Cry4Ba toxin of Bacillus thuringiensis israelensis (Bti) and that fragments of AgCad1 synergizes Cry toxicity to larvae of the genera Anopheles, Aedes and Culex mosquitoes.

To summarize some of the work reported herein, a protein with some similarity to lepidopteran cadherins was identified in An. gambiae databases, and the corresponding cadherin cDNA was cloned. The cDNA encodes a 195-kDa protein with a predicted leader peptide, 11 cadherin repeats, a membrane-proximal extracellular domain, a membrane spanning region, and an internal cytoplasmic domain. Anti-serum prepared against E. coli-expressed cadherin, detected a 210-kDa protein in brush border membrane preparations. The cadherin-like protein, as visualized by immunohistochemistry of sectioned gut material, was localized in the posterior midgut on the apical portion of the brush border. Bti toxins were examined for their ability to bind An. gambiae cadherin. Cry4Ba toxin bound 210-kDa cadherin on blots of larval brush border protein. Rhodamine-labeled Cry4Ba toxin co-localized with cadherin on the microvilli of sectioned midgut tissue. Under non-denaturing conditions, Cry4Ba toxin bound cadherin expressed in Drosophila-S2 cells and binding was specific and competitive. Thus, we identified this An. gambiae cadherin protein as a putative receptor for Cry4Ba toxin.

More specifically, a midgut cadherin AgCad1 cDNA was cloned from Anopheles gambiae larvae and was analyzed for its possible role as a receptor for the Cry4Ba toxin of Bacillus thuringiensis strain israelensis. AgCad1 in the larval brush border is identified herein as a binding protein for Cry4Ba toxin. Although Cry4Ba showed limited binding to CR11-MPED of AgCad1, the peptide synergized the toxicity of Cry4Ba to larvae.

The AgCad1 cadherin encodes a 1735-residue protein organized into an extracellular region of 11 cadherin repeats (CR) and a membrane-proximal extracellular domain (MPED). AgCad1 mRNA was detected in midgut of larvae by polymerase chain reaction (PCR).

The AgCad1 protein was localized, by immunochemistry of sectioned larvae, predominately to the microvilli in posterior midgut. The localization of Cry4Ba binding was determined by the same technique and toxin bound microvilli in posterior midgut. The AgCad1 protein was present in brush border membrane fractions prepared from larvae and Cry4Ba toxin bound the same-sized protein on blots of those fractions.

The AgCad1 protein was expressed transiently in Drosophila melanogaster Schneider 2 (S2) cells. ¹²⁵I-Cry4Ba toxin bound AgCad1 from S2 cells in a competitive manner. Cry4Ba bound to beads extracted 200-kDa AgCad1 and a 29-kDa fragment of AgCad1 from S2 cells. Thus, cadherin expressed on Dm-S2 cells was specifically bound to Cry4Ba.

A peptide containing the AgCad1 region proximal to the cell (CR11-MPED; SEQ ID NOs:3 and 4) was expressed in Escherichia coli. Although Cry4Ba showed limited binding to CR11-MPED, the peptide synergized the toxicity of Cry4Ba to larvae.

AgCad1 in the larval brush border is a binding protein for Cry4Ba toxin. Based on binding results and CR11-MPED synergism of Cry4Ba toxicity, AgCad1 is probably a Cry4Ba receptor.

The cloning and identification of a cadherin-like protein from the gut of An. gambiae is described herein. Bioassays, immunohistochemistry, and toxin binding studies are utilized to characterize this cadherin protein, the first reported function of a cadherin as a putative Bt toxin receptor in mosquito larvae.

In this study, we present analysis of a cadherin-like protein, AgCad1, which is expressed in the midgut of An. gambiae larvae. We present supporting data to show that AgCad1 is a binding protein and possibly a functional receptor for Cry4Ba toxin. AgCad1 bound Cry4Ba toxin in BBMV prepared from larvae and when expressed in S2 cells. A truncated fragment of AgCad1, CR11-MPED, enhanced Cry4Ba toxicity to the mosquito larvae.

Some midgut cadherins function as Cry1A toxin receptors (e.g. Bt-R₁) in lepidopteran larvae. Bt-R₁ is located on the apical membrane of midgut columnar epithelial cells (Chen, J. et al. 2005. Cell Tissue Res. 321: 123-9), unlike classical cadherins, which are located mainly within cadherin junctions involved in cell-cell adhesion (Angst, B. D. et al. 2001. J. Cell Sci. 114: 629-41).

The subject mosquito cadherin was also localized on the apical membrane in the gut region of the larva. Previous research shows that the apical region of the posterior gut in An. gambiae binds Cry4A protein (Ravoahangimalala, O. and Charles, J. F. 1995. FEBS Lett. 362: 111-115). We also localized Cry4Ba binding to the brush border of the posterior gut. This pattern of binding correlates with the presence of receptors.

The AgCad1 protein has features expected of a member of the cadherin superfamily. AgCad1 has 11 cadherin repeats compared to 12 cadherin repeats in Bt-R₁. Also, both cadherin proteins contain an MPED followed by a predicted membrane spanning region. Similar to lepidopteran cadherins, the cytoplasmic domain of AgCad1 does not have sequences predicted to interact with intracellular proteins such as catenins AgCad1 has 29% identity with Bt-R₁ in pair-wise alignment. A paralogue of AgCad1 in An. gambiae (PCAP; XM_{—}321513.2; SEQ ID NOs:7 and 8) shows 18% identity and an orthologue in D. melanogaster cad88C/15646 shows 17% identity. The function of cad88C is not reported in the literature. Bel and Escriche (2006. Gene 381: 71-80) noted that in zebrafish and mammals an orthologue of lepidopteran midgut cadherins, cadherin 23, is involved in maintenance of hair bundles (stereocilia) of the inner ear, related to signal mechanotransduction.

AgCad1 was detected as a 200 kDa protein in BBMV prepared from An. gambiae larvae. Although the same-sized protein bound Cry4Ba on ligand blots, Cry4Ba did not bind to S2 cell-expressed AgCad1 on ligand blots. This was the case when S2 cell protein was either run directly on blots, or enriched by partial-purification. In contrast Cry4Ba bound partially purified AgCad1 in dot blot experiments, and binding was competed by unlabeled toxin. The dot-blot results and the observation that Cry4Ba extracted non-denatured AgCad1 from S2 cells, suggests that secondary structure of AgCad1 may contribute to Cry4Ba binding. There is precedence for this explanation as Cry1Ab binds a motif on Bt-R₁ comprised of the N- and C-terminal ends of Bt-R₁ brought together by secondary structure (Griko, N. B. et al. Biochemistry 46: 10001-10007).

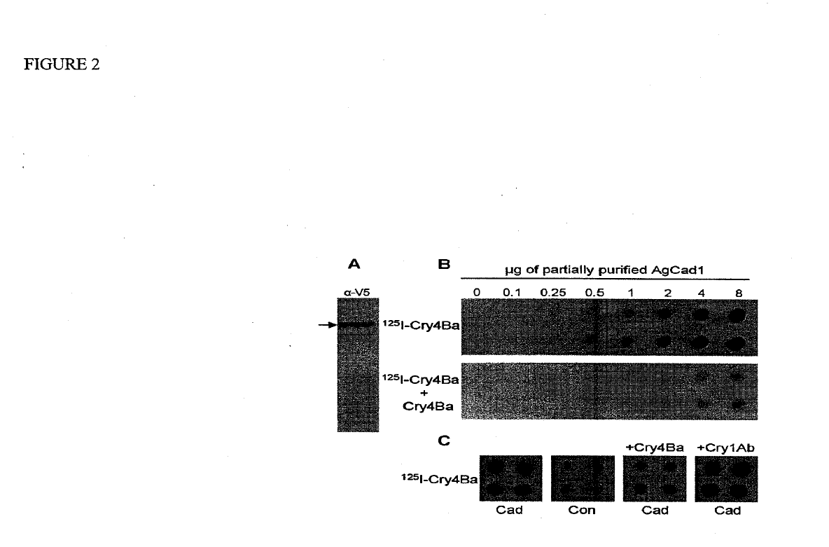

The CR11-MPED region of AgCad1 bound Cry4Ba toxin on dot blots; however the binding signal was considerably weaker than seen with the full-length AgCad1 and competition by unlabeled Cry4Ba was less obvious. In contrast, the comparable peptide, CR12-MPED from Bt-R1, gave a strong signal on dot blots and bound toxin at a high affinity site (Kd=9 nM) and low affinity sites (Kd=1 μM) (Chen, J. et al. 2007. Proc. Natl. Acad. Sci. U.S.A. 104: 13901-13906). Although we were unable to quantify the dot blot binding data shown in FIG. 2, and calculate the affinity value, qualitatively the data suggest that the affinity of Cry4Ba for the CR11-MPED peptide is much lower than the affinity of Cry1Ab for CR12-MPED from Bt-R₁. Xie et al. (2005. J. Biol. Chem. 280: 8416-8425) determined the Cry1Ac binding motif in H. virescens cadherin as GVLTLNFQ (SEQ ID NO:15), which is located in the last repeat of the cadherin. A similar region, GVLTLNIQ (SEQ ID NO:16), present in M. sexta Bt-R₁, affects binding and Cry1A toxicity on lepidopteran larvae (23). CR12-MPED-mediated Cry1A toxin enhancement was significantly reduced when the high affinity Cry1A binding epitope (GVLTLNIQ) (SEQ ID NO:16) within the cadherin peptide was deleted. It is interesting to note that a similar conserved region, GELTLTSKVQ (SEQ ID NO:17), is located within the last repeat of An. gambiae cadherin molecule.

Enhancement of Cry4Ba toxicity to An. gambiae larvae by CR11-MPED indicates that the toxicity enhancement properties of cadherin fragments extends at least to Cry toxins active against dipteran larvae. Overall, the data also demonstrates that midgut cadherin, AgCad1, is a Cry4Ba binding protein and putative receptor. Further investigations of the interaction of Cry4Ba, and other mosquitocidal Cry toxins, with midgut molecules can be conducted to further characterize the role of midgut cadherin in the intoxication process.

Without being bound by a specific theory or theories of mechanism of action, one possibility is that these fragments work in conjunction with B.t. toxins and enhance the pesticidal activity of the toxin. When fed to insects with a Cry toxin, the peptide can change the effect of a toxin from a growth-inhibitory effect to an insecticidal effect. In addition or alternatively, the fragments can exert at least a partial toxic effect by a separate mechanism of action. Yet another possibility is that the fragments also, or alternatively, work indirectly to stabilize the B.t. toxin. Thus, said fragment can work independently from the Cry toxin (by another mechanism of action) and/or in conjunction with the Cry toxin to enhance the insecticidal potency of the Cry toxin. Again, Cry binding to the cadherin fragment (comprising a Cry binding domain) may promote the switch of toxin from monomer to oligomer according to the Bravo model (Bravo, A. et al. 2004. Biochim Biophys Acta 1667: 38-46). However, the subject invention can be practiced without a full understanding of the underlying mechanism(s) of action.

It will be recognized by those skilled in the art that DNA sequences of the subject invention may vary due to the degeneracy of the genetic code and codon usage. All DNA sequences which code for exemplified and/or suggested peptides (and proteins) are included. For example, the subject peptides are included in this invention, including DNA (optionally including an ATG preceding the coding region) that encodes the CR11 region (to and optimally including the MPED region) of SEQ ID NOs:1 and 2. Fragments of SEQ ID NO: 4 and SEQ ID NO: 6, for example, should include a Cry binding site for use according to the subject invention. The subject invention also includes polynucleotides having codons that are optimized for expression in plants, including any of the specific types of plants referred to herein. Various techniques for creating plant-optimized sequences are known in the art.

Additionally, it will be recognized by those skilled in the art that allelic variations may occur in the DNA sequences which will not significantly change activity of the amino acid sequences of the peptides which the DNA sequences encode. All such equivalent DNA sequences are included within the scope of this invention and the definition of the regulated promoter region. The skilled artisan will understand that exemplified sequences (such as the CR11 and CR11-MPED fragments of SEQ ID NOs:1 and 2) can be used to identify and isolate additional, non-exemplified nucleotide sequences which will encode functional equivalents to the sequences given in, or an amino acid sequence of greater than 90% identity thereto and having equivalent biological activity. DNA sequences having at least 90%, or at least 95% identity to a recited DNA sequence and encoding functioning peptides (such as CR-11/CR11-MPED) are considered equivalent sequences and are included in the subject invention. Other numeric ranges for variant polynucleotides and amino acid sequences are provided below (e.g., 50-99%). Following the teachings herein and using knowledge and techniques well known in the art, the skilled worker will be able to make a large number of operative embodiments having equivalent DNA sequences to those listed herein without the expense of undue experimentation.

As used herein percent sequence identity of two nucleic acids is determined using the algorithm of Karlin and Altschul (1990. Proc. Nod Acad. Sci. USA 87: 2264-2268) modified as in Karlin and Altschul (1993. Proc. Natl. Acad. Sci. USA 90:5873-5877). Such an algorithm is incorporated into the NBLAST and XBLAST programs of Altschul et al. (1990. J. Mol. Biol. 215: 402-410). BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997. Nucl. Acids. Res. 25:3389-3402). When utilizing BLAST and Gapped BLAST programs, the default parameters of the respective programs (NBLAST and XBLAST) are used. See ncbi.nih.gov website.

Polynucleotides (and the peptides and proteins they encode) can also be defined by their hybridization characteristics (their ability to hybridize to a given probe, such as the complement of a DNA sequence exemplified herein). Various degrees of stringency of hybridization can be employed. The more stringent the conditions, the greater the complementarity that is required for duplex formation. Stringency can be controlled by temperature, probe concentration, probe length, ionic strength, time, and the like. Preferably, hybridization is conducted under moderate to high stringency conditions by techniques well known in the art, as described, for example, in Keller, G. H., M. M. Manak (1987. DNA Probes, Stockton Press, New York, N.Y., pp. 169-170).

As used herein “moderate to high stringency” conditions for hybridization refers to conditions that achieve the same, or about the same, degree of specificity of hybridization as the conditions “as described herein.” Examples of moderate to high stringency conditions are provided herein. Specifically, hybridization of immobilized DNA on Southern blots with ³²P-labeled gene-specific probes was performed using standard methods (Maniatis et al.). In general, hybridization and subsequent washes were carried out under moderate to high stringency conditions that allowed for detection of target sequences with homology to sequences exemplified herein. For double-stranded DNA gene probes, hybridization was carried out overnight at 20-25° C. below the melting temperature (Tm) of the DNA hybrid in 6×SSPE, 5×Denhardt's solution, 0.1% SDS, 0.1 mg/ml denatured DNA. The melting temperature is described by the following formula from Beltz et al. (1983).

Tm=81.5° C.+16.6 Log [Na+]+0.41(% G+C)−0.61 (% formamide) 600/length of duplex in base pairs.

Washes are typically carried out as follows:
(1) Twice at room temperature for 15 minutes in 1×SSPE, 0.1% SDS (low stringency wash).
(2) Once at Tm-20° C. for 15 minutes in 0.2×SSPE, 0.1% SDS (moderate stringency wash).

For oligonucleotide probes, hybridization was carried out overnight at 10-20° C. below the melting temperature (Tm) of the hybrid in 6×SSPE, 5×Denhardt's solution, 0.1% SDS, 0.1 mg/ml denatured DNA. Tm for oligonucleotide probes was determined by the following formula from Suggs et al. (1981):

Tm (° C.)=2 (number T/A base pairs)+4(number G/C base pairs)

Washes were typically carried out as follows:
(1) Twice at room temperature for 15 minutes 1×SSPE, 0.1% SDS (low stringency wash).
(2) Once at the hybridization temperature for 15 minutes in 1×SSPE, 0.1% SDS (moderate stringency wash)

In general, salt and/or temperature can be altered to change stringency. With a labeled DNA fragment of greater than about 70 or so bases in length, the following can be used:

Low: 1 or 2×SSPE, room temperature

Low: 1 or 2×SSPE, 42° C.

Moderate: 0.2× or 1×SSPE, 65° C.

High: 0.1×SSPE, 65° C.

Duplex formation and stability depend on substantial complementarity between the two strands of a hybrid, and, as noted above, a certain degree of mismatch can be tolerated. Therefore, polynucleotide sequences of the subject invention include mutations (both single and multiple), deletions, and insertions in the described sequences, and combinations thereof, wherein said mutations, insertions, and deletions permit formation of stable hybrids with a target polynucleotide of interest. Mutations, insertions, and deletions can be produced in a given polynucleotide sequence using standard methods known in the art. Other methods may become known in the future.

The mutational, insertional, and deletional variants of the polynucleotide and amino acid sequences of the invention can be used in the same manner as the exemplified sequences so long as the variants have substantial sequence similarity with the original sequence. As used herein, substantial sequence similarity refers to the extent of nucleotide similarity that is sufficient to enable the variant polynucleotide to function in the same capacity as the original sequence. Preferably, this similarity is greater than 50%; more preferably, this similarity is greater than 75%; and most preferably, this similarity is greater than 90%. The degree of similarity needed for the variant to function in its intended capacity will depend upon the intended use of the sequence. It is well within the skill of a person trained in this art to make mutational, insertional, and deletional mutations that are designed to improve the function of the sequence or otherwise provide a methodological advantage. The identity and/or similarity can also be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein.

The amino acid identity/similarity and/or homology will be highest in critical regions of the protein that account for biological activity and/or are involved in the determination of three-dimensional configuration that ultimately is responsible for the biological activity. In this regard, certain amino acid substitutions are acceptable and can be expected if these substitutions are in regions that are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Following is a list of examples of amino acids belonging to each class.

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

In some instances, non-conservative substitutions can also be made. The critical factor is that these substitutions must not significantly detract from the biological activity of the toxin.

As one skilled in the art will appreciate in light of the subject disclosure, formulations for delivering Bti biopesticides can be adapted for use according to the subject invention. The performance of Bti biopesticides relies in part on the ingestion of the crystals by mosquito larvae. Therefore, different types of Bti formulations are used to control mosquitoes in different habitats. Common formulations are granular, flowable or even slow-release for control of container breeding mosquitoes. Surface-feeding Anopheles species are best-controlled by formulations that float on the water surface. There has been some development of incorporating Bti crystals into ‘ice granules.’ Recombinant applications of Bti cry genes include engineering into Bacillus thuringiensis, Bacillus sphaericus, E. coli, the protozoan Tetrahymena pyriformis and rice plants. In each case the goal is to control a dipteran insect by producing a Cry toxin in a microorganism that is introduced into the larval habitat where it is ingested. There has also been development of non-viable recombinant organisms that could increase persistence in the environment, such as products based on encapsulated Bt toxins in Pseudomonas fluorescens. This approach ameliorates concerns associated with releasing live genetically engineered microorganisms into the environment.

In some preferred embodiments, the subject peptides are fed to target insects together with one or more insecticidal proteins, preferably (but not limited to) B.t. Cry proteins. When used in this manner, the peptide fragment can not only enhance the apparent toxin activity of the Cry protein against the insect species that was the source of the receptor but also against other insect species.

A related aspect of the inventions pertains to the use of an isolated polynucleotide that encodes a protein comprising (or consisting of) a fragment of a cadherin-like protein. The subject invention includes a cell (and use thereof) carrying the polynucleotide and expressing the peptide fragment, including methods of feeding the peptide (preferably with B.t. Cry toxins) to insects.

As used herein, reference to “isolated” polynucleotides and/or “purified” proteins refers to these molecules in other-than a state of nature. Thus, reference to “isolated” and/or “purified” signifies the involvement of the “hand of man” as described herein. For example, a “gene” of the subject invention put into a plant for expression is an “isolated polynucleotide.”

The nucleotide sequences can be used to transform bacterial hosts for the purpose of producing the cadherin fragments. Such bacterial hosts may include Bacillus thuringiensis, Bacillus sphaericus, Escherichia coli and Pseudomonas fluorescens. In some embodiments the cells would be lysed and the cadherin protein extracted or the lysate may be used, preferably with Bti Cry proteins, for insect control. In some embodiments, the cadherin fragment expressed in bacterial cells would be used without killing or lysing the cells. Microorganisms other than bacteria could be used in this manner.

The nucleotide sequences can be used to transform hosts, such as plants, to express the receptor fragments (preferably cadherin fragments) of the subject invention. Transformation of plants with the genetic constructs disclosed herein can be accomplished using techniques well known to those skilled in the art. Thus, in some embodiments, the subject invention provides nucleotide sequences that encode fragments of receptors, preferably AgCad1, AgPCAP or Bt-R₁ cadherin-like protein. Production of the cadherin protein in leaves or stems could utilize constitutive promoters such as the 35S promoter or T-DNA promoters which are well-known in the art.

Alternatively, promoters could be selected that direct expression of the cadherin fragment to the seed. The napin promoter (napA) of Brassica napus is an example of an endosperm-specific promoter of this type (Ellerstrom et al. 1996. Plant Molec. Biol. 32:1019-1027. Protein production in cereal grains such as rice or barley is also a means to produce large amounts of the cadherin fragment for insect control. The globulin promoter of rice is suitable for high level protein production in rice (Hwang et al. 2002. Plant Cell Rep. 20: 842-847). Plants containing the expressed cadherin fragment could be ground into meal, mixed with Cry proteins and delivered to the habitat of the pest larvae for insect control. Seeds of plants containing the expressed cadherin fragment could be ground into plant flour, mixed with Cry proteins and delivered to the habitat of the pest larvae for insect control.

Cadherin fragments could be co-expressed in plants alone or with one or more Bt Cry proteins. Additionally, more than one type of cadherin fragment could be selected for co-expression in plants.

The receptor used as the source of this domain(s), for use against dipterans, can be derived from various pests and insects, particularly dipterans such as Anopheles gambiae and Aedes aegypti. However, fragments of the subject invention could also be derived from midgut, Cry-binding cadherins from non-dipteran insects, such as Manduca sexta larvae. Many sequences of such receptors are publicly available.

Dipterans are the preferred target pest according to the subject invention. Various dipterans can be targeted, including but not limited to Anopheles gambiae, Aedes aegypti and Culex pipiens. Flies, including Black flies in the genus Simulium and fungus gnats in the genus Orefelia, may also be targeted with the subject invention. Sandflies, in the genera Phlkebotomus, Sergentomyia and Lutzomya could be targeted with this invention. Dipteran, including those in the genus Tipula, which are pests of grasslands and pastures, could be targeted with the subject invention. Midges in the genus Chironimus, pests of rice, can be targeted with the subject invention. The suborder Nematocera is also significant.

Because of the unique and novel approach of the subject invention, dipteran pests that were typically not susceptible to Bt. Cry proteins can now also be targeted. The subject invention can be used to enhance and expand the spectrum (or insect range) of toxicity of a given insect-toxic protein.

In some preferred embodiments, these peptide fragments can be used to enhance the potency of B.t. toxins for controlling insects. In some preferred embodiments, the peptide fragments enhance the toxicity of Cry1 toxins, but as shown herein, the subject invention is not limited to use with such toxins.

Based on the subject disclosure, one skilled in the art can practice various aspects of the subject invention in a variety of ways. For example, the fragment of cadherin-like protein may be expressed as a fusion protein with a B.t. Cry toxin using techniques well known to those skilled in the art. As described herein, preferred fusions would be chimeric toxins produced by combining a toxin (including a fragment of a protoxin, for example) and a fragment of a cadherin-like protein. In addition, mixtures and/or combinations of toxins and cadherin-like protein fragments can be used according to the subject invention. These mixtures or chimeric proteins have the unexpected and remarkable properties of enhanced insecticidal potency to dipteran larvae.

All patents, patent applications, provisional applications, and publications referred to or cited herein are incorporated by reference in their entirety to the extent they are not inconsistent with the explicit teachings of this specification.

Unless specifically indicated or implied, the terms “a”, “an”, and “the” signify “at least one” as used herein.

Following are examples that illustrate procedures for practicing the invention. These examples should not be construed as limiting. All percentages are by weight and all solvent mixture proportions are by volume unless otherwise noted.

### EXAMPLE 1

### Materials and Methods

Insects. An. gambiae (CDC G3 strain) were maintained at 27° C. with a photoperiod of 14 h light: 10 h dark. Larvae (ca. 200/pan) were fed ground fish food (TetraMin) daily. Adults were fed with 10% sucrose solution and females were fed on anesthetized mice until engorged. Freshly laid eggs were collected, washed with 0.1% bleach and hatched in distilled water. Larvae were grown until 4th instar, collected and stored at −80° C. until use.

Primers. The sequences of primers used for cloning in this study are listed in Table 1.

Table 1. The primers used in the PCR cloning of AgCad1, the cloning of cadherin fragments CR11-MPED and TM-cyto and the PCR analysis of AgCad1 and AgPCAP in Anopheles gambiae larvae.

| | |
|---|---|
| Primer | Primer Sequence (5′-3′) |

Internal Gene Specific Primer

| | |
|---|---|
| AgCad/F1 | 5′-GGT GGC CGC TGG TCG ATC GTA ATC |
| | AAT CGC CG-3′ |
| | (SEQ ID NO: 18) |
| AgCad/R1 | 5′-CTT AAT TTT CAG TGT CCA CGT TCC |
| | GTA AAA TCC-3′ |
| | (SEQ ID NO: 19) |

3' RACE Primers

| | |
|---|---|
| AgCad/F2 | 5′-CGT GTA TCG TTC ACG ATC AAC ATC |
| | AAC AAT GCG-3′ |
| | (SEQ ID NO: 20) |
| AgCad/F3 | 5′-ATC ATC GCT CAC GAC ATT GAC GGA |
| | CCA GG-3′ |
| | (SEQ ID NO: 21) |

5' RACE Primers

| | |
|---|---|
| AgCad/R3 | 5′-GCA CCC TCG CTG GAG GTG TTC AGC |
| | AGC CGG TT-3′ |
| | (SEQ ID NO: 22) |
| AgCad/R2 | 5′-GGT CCC GCG CAC CGG CCA CAT CAC |
| | CGA TCT CG-3′ |
| | (SEQ ID NO: 23) |

Primers for cloning 5′ and 3′ fragments of cDNA

| | |
|---|---|
| AgCad/F-Spe | 5′-GTT ACC TAG TGT ACC GGC TGC TGG |
| | CGG CCT TAA-3′ |
| | (SEQ ID NO: 24) |
| AgCad/R-Bam | 5′-CGT TCG TCT CAG CGC CCG GGG GAA |
| | GGC CCG C-3′ |
| | (SEQ ID NO: 25) |
| AgCad/F-BamH | 5′-CCG TTT GCC GAG GAT CCG AAG AAC |
| | GCG GGC-3′ |
| | (SEQ ID NO: 26) |
| AgCad/R-Sac | 5′-GAA TTC GCG GCC GCG GGA ATT TTT |
| | TTT TTT TTT TTT-3′ |
| | (SEQ ID NO: 27) |

For cloning CR11-MPED

| | |
|---|---|
| CR11-MPED/F | 5′-GAC CCA TAT GGA CGA AAC GCT GCA |
| | GAT CAT CCT GA-3′ |
| | (SEQ ID NO: 28) |
| CR11-MPED/R | 5′-ACA CCT CGA GGA ACC GGT GGG ACA |
| | GCT CGT CGT CA-3′ |
| | (SEQ ID NO: 29) |

For cloning TM-Cyto

| | |
|---|---|
| TM-Cyto/F | 5′-GAC CCA TAT GGA CGA AAC GCT GCA |
| | GAT CAT CCT GA-3′ |
| | (SEQ ID NO: 28) |
| TM-Cyto/R | 5′-ACA CCT CGA GGA ACC GGT GGG ACA |
| | GCT CGT CGT CA-3′ |
| | (SEQ ID NO: 29) |

For PCAP PCR

| | |
|---|---|
| AgPCAP/F | 5′-GGT ATC TCA ACG TCG TCG CTG-3′ |
| | (SEQ ID NO: 30) |
| AgPCAP/R | 5′-CCTCCAGCACGGAGTTGTTC-3′ |
| | (SEQ ID NO: 31) |

Synthesis of cDNA and cloning An. gambiae cadherin AgCad1. RNA was extracted from A. gambiae 4^{th} instar larvae (75 mg wet weight) using the total RNA mini kit (Bio-Rad). First strand cDNA was synthesized from total RNA with oligo-dT₁₇ primer, dNTPs and SuperScript reverse transcriptase II (Invitrogen) according to the manufacturer. A pair of primers, AgCad1/F1 and AgCad1/R1 (Table 1), was designed to match the ends of the partial sequence of A. gambiae cadherin (GenBank XM_{—}312086). PCR products were amplified using synthesized cDNA as template and cloned into pGEM-T easy vector (Promega). The DNA inserts were sequenced in both forward and reverse directions at the Molecular Genetics Instrumentation Facility at University of Georgia confirming the cloned cDNA as identical to A. gambiae cadherin sequence (XM_{—}312086).

For 3′ rapid amplification of cDNA ends (RACE), An. gambiae cDNA was synthesized from total RNA using Not I-d(T)₁₇ primer and SuperScript reverse transcriptase. The cDNA was amplified by PCR with AgCad1/F1 and Not I-d(T)₁₇ as primers. The PCR product was further amplified with first round primers AgCad1/F2 and Not I-d(T)₁₇. The resultant PCR product was then subjected to a second round amplification with the nested primer AgCad1/F3 and Not I-d(T)₁₇. The product was purified, cloned into pGEM-T easy vector (Promega) and then sequenced.

The 5′ end of the cadherin region was amplified with the Gibco-BRL 5′ RACE kit and two gene-specific primers (GSPs) AgCad1/R2 and AgCad1/R3. SuperScript reverse transcriptase was used to synthesize first strand cDNA with GSP1 (AgCad1/R1). The resultant cDNA was then used as template for amplification with GSP2 (AgCad1/R2) and oligo-dG abridged anchor primer (Invitrogen). The PCR product from the AgCad1/R2 reaction was purified, cloned into plasmid pGEM-T Easy (Promega) and sequenced.

Bioinformatic analysis. Bioinformatic analysis using ISREC ProfileScan server (website hits.isb-sib.ch/cgi-bin/PFSCAN) was performed to analyze the full cadherin sequence. The software basically performs computational predictions using protein sequence patterns (or motifs) from known, well characterized proteins in the database to elucidate the potential function(s) of uncharacterized proteins (Sigrist, C. J., et al. 2002. Brief Bioinform. 3: 265-274.

PCR detection of AgCad1 mRNA in larval gut tissue. Twenty 4th instar larvae were placed in RNAlater™ (Sigma) for fixation and dissection. While observing under a dissecting scope, the whole intestine was gently pulled out using fine forceps. After removing Malphigian tubules, dissected guts were immediately used for cDNA synthesis. Methods for cDNA synthesis were the same as described above for larval cDNA synthesis. Plasmids pIZT-AgCad1, pIZT-AgPCAP and gut cDNA served as templates for PCR. Primers CR11-MPED/F and CR11-MPED/R served as AgCad1-specific primers. As a control, primers AgPCAP/F and AgPCAP/R were designed to amplify a region from a second cadherin-like gene in An. gambiae [putative cell adhesion protein (PCAP); Genbank: AJ439060]. PCR was performed with 30 cycles of 94° C. for 30 sec, 55° C. for 30 sec and 72° C. for 40 sec and the products were separated on a 1% agarose gel.

Assembling and cloning cadherin gene into pIZT/V5 insect cell expression vector. PCR was conducted using Not I-d(T) cDNA as a template with primers AgCad1/F-Spe and AgCad1/R-BamH. Long-template polymerase (Roche Applied Science) was used in a PCR with 30 cycles of 94° C. for 2 min and 68° C. for 4 min. The resultant PCR fragment was purified and then cleaved with SpeI and BamHI followed by cloning into pMECA plasmid vector (GenBank AF017063) (25) yielding the 5′ cadherin clone called pMECA-AgCad1-5′. Oligonucleotide primers AgCad1/F-BamH and AgCad1/R-Sac were used to amplify the 3′-end of the AgCad1 coding region using the cDNA template, using an Expand Long Template PCR System (Roche Applied Science) with 30 cycles of 94° C. for 2 min and 68° C. for 2 min. The PCR fragment was extracted from an agarose gel, digested with BamHI and SacII, and then cloned into pMECA vector to obtain pMECA-AgCad1-3′. Both 5′ and 3′ clones were sequenced in forward and reverse directions.

The DNA insert in pMECA-AgCad1-3′ was excised by digestion with BamHI and SacII and cloned into pMECA-AgCad1-5′ treated with the same two restriction enzymes, yielding pMECA-AgCad1. The full-length cadherin coding region was excised from pMECA-AgCad1 with SpeI and SacII, purified and cloned into plasmid pIZT (Invitrogen) previously digested with the same enzymes. Fidelity of the full-length cadherin in plasmid pIZT was confirmed by DNA sequencing and the plasmid was named pIZT-AgCad1.

Transient Expression of An. gambiae cadherin in Drosophila S2 cells. Drosophila melanogaster-Drosophila melanogaster (Dm) S2 cells (Invitrogen) were cultured in serum-free insect cell medium (HyClone, Logan, Utah). For plasmid transfection, fresh S2 cells (1.5×10⁶) were seeded into a 60 mm² polystyrene culture dish and allowed to adhere overnight. Plasmid transfection mixtures consisted of pIZT (5 μg) or pIZT-AgCad1 (10 μg) in 1 ml of culture medium plus 10 μl Cellfectin reagent (Invitrogen). Each transfection mixture was pre-incubated at room temperature for 30 min, transferred to a dish containing S2 cells and the dishes incubated with gentle shaking for 4 hours. Fresh medium (5 ml) was added to the dish after removal of the transfection mixtures, and S2 cells were incubated at 25° C. for 3 days.

Preparation of brush border membrane vesicles (BBMV) from An. gambiae larvae. BBMV were prepared from whole 4^{th} instar larvae according to Abdul-Rauf Ellar (1999. J. Invertebr. Pathol. 73: 45-51) with slight modifications. Eight grams of larvae were homogenized in 100 ml ice cold MET buffer (300 mM mannitol, 5 mM EGTA, 17 mM Tris, pH 7.5) containing Complete™ cocktail protease inhibitor (Roche Applied Science) for 1 min using a tissue homogenizer (Kinematica GmbH) set at the highest speed, followed by further homogenization with 15-20 strokes of a Dounce homogenizer. An equal volume of ice-cold 24 mM MgCl₂ was mixed with the homogenate and the mixture placed on ice for 15 min. The mixture was centrifuged at 1900 g for 15 min at 4° C., and the supernatant further centrifuged at 27000 g for 30 min. The resulting pellet was homogenized in MET buffer, mixed with an equal volume of 24 mM MgCl₂, and centrifuged at low and high speed as above. The final pellet was re-suspended in 3 ml of ice-cold MET buffer with protease inhibitors. Protein amount was determined by the Bio-Rad protein assay (Bio-Rad) with BSA as standard. Aminopeptidase N activity (Garczynski, S. F., and Adang, M. J. 1995. Insect Biochem. Mol. Biol. 25: 409-415), a marker for brush border membranes, was enriched about 6-fold for the final BBMV preparation compared to the initial crude larval homogenate (data not shown).

Cloning and expression of An. gambiae CR11-MPED and TM-Cyto peptides. A partial cadherin peptide (amino acids 1358G to 1569A) spanning domains CR11-MPED) was over-expressed in E. coli as inclusion bodies. The plasmid pMECA-AgCad1-3′ was used as a template to amplify the region encoding CR11-MPED by PCR with CR11-MPED/F and CR11-MPED/R primers. The resulting PCR fragment was cloned into the pET-30a(+) vector (Novagen) to yield plasmid pET-AgCad1/CR11-MPED. After confirmation by sequencing, plasmid pET-AgCad1/CR11-MPED was transformed into E. coli strain BL21-CodonPlus (DE3)/pRIL (Stratagene). The CR11-MPED region was over-expressed by induction with 1 mM isopropyl β-D-thiogalactopyranoside (IPTG) when the culture OD600 reached 0.5-0.6. The expression and purification protocols are described in a previous paper (Chen et al. 2007. Proc. Natl. Acad. Sci. U.S.A. 104: 13901-13906). Polyclonal α-serum against purified CR11-MPED, referred to as anti-AgCad1 serum, was produced in New Zealand White rabbits at the Animal Resources Facility at the University of Georgia.

A cadherin truncation (amino acids 1570D to 1735F) containing predicted transmembrane (TM) and cytoplasmic (Cyto) domains was also subcloned to pET-30a(+) vector to yield pET-AgCad1/TM-Cyto by PCR with primer TM-Cyto/F and TM-Cyto/R.

Immunohistochemistry and Cry4Ba binding localization. Dissected guts of early 4^{th} instar larvae of A. gambiae were fixed with 4% paraformaldehyde in PBS for 2 h on ice. Fixed tissues were soaked in 30% sucrose solution overnight at 4° C. and embedded in a capsule containing Tissue Freezing Medium (Triangle Biomedical Sciences). The capsule was snap-frozen in liquid nitrogen and the block transferred immediately into the chamber of a cryostat (Reichert-JuAg 2800 Frigocut-E cryostat). Sections (10 microns) of embedded guts were cut serially and mounted on Superfrost/Plus slides (Fisher Scientific).

Slides containing air-dried tissue sections were washed with PBS for 20 min, blocked with 1 ml PBST-5% BSA buffer (PBS solution with 0.2% Tween-20 and 5% BSA) for 1 h at room temperature. Subsequent steps of immuno-detection, toxin binding and observation were according to Chen et al. (2005. Cell Tissue Res. 321: 123-129). Cadherin was detected by anti-AgCad1 serum diluted 1:500 in blocking solution, pre-immune serum diluted in blocking solution served as a negative control. To detect Cry4Ba binding, tissue sections were treated with 5 μg/ml rhodamine-labeled [rhodamine derivative, 5-(6)-carboxy-tetramethylrhodamine (TAMRA)]-Cry4Ba. Rhodamine-labeled BSA (5 μg/ml) was used as a control.

Immuno and toxin blots. A. gambiae BBMV proteins were separated by SDS-PAGE and electroblotted to PVDF filters. Filters were blocked with 3% bovine serum albumin (BSA) in PBST (PBS+0.1% Tween 20) for 1 hour at room temperature, and then probed with α-AgCad1 serum (1:5000 dilution) in PBST/0.1% BSA for 2 h. After washing, the filters were incubated with α-rabbit IgG-peroxidase conjugate (1:25,000 dilution) in the same buffer for 1 h at room temperature. Finally, the filters were developed with an ECL kit (GE Healthcare) and exposed to X-ray film. To detect cadherin expression in S2 cells, 1×10⁷ cells were harvested by centrifugation at 400 g for 2 min followed by three washes with PBS. Whole cells were suspended in SDS-PAGE sample buffer and boiled for 10 min. Expressed cadherin on S2 cells was detected on western blots using anti-AgCad1 serum.

A. gambiae BBMV were treated with Plus-One 2-D Clean-up kit (GE Healthcare) according to the manufacturer's instructions and 20 μg protein was separated by SDS-PAGE. After electrophoresis, separated proteins were transferred to a PVDF filter and blocked with 3% BSA in PBST. The filter was incubated with Cry4Ba (5 μg/ml final concentration) in PBST for 1 h at room temperature. Toxin binding proteins were detected with rabbit α-Cry4Ba serum, and developed by an ECL kit (GE Healthcare).

Cry4Ba-bead extraction of AgCad1 expressed in S2 cells. S2 cells expressing cadherin were harvested and washed as described above. The cells were suspended and solubilized in PBS containing 1% CHAPS with cocktail protease inhibitor (Roche Applied Science) with rotation at room temperature for 1 h. Solubilized proteins were clarified by centrifugation at 16200 g for 30 min. The proteins were mixed with a Cry4Ba-anti-Cry4Ba conjugated Protein-A bead column. Protein-A Sepharose™ 6MB (GE Healthcare) beads were washed with PBS three times and then incubated with α-Cry4Ba serum plus varying amounts of Cry4Ba with rotation for 1 h at room temperature. Beads without Cry4Ba-anti-Cry4Ba conjugate were used as background control.

The solubilized S2 cell proteins were incubated with the Cry4Ba-α-Cry4Ba/Protein-A bead complex for 2 h at room temperature with rotation. The beads were pelleted by centrifugation at 100 g for 1 min and vigorously washed three times with PBS. The sample was then heated in a 100° C. bath for 10 min with SDS-sample buffer to extract the bound proteins. Proteins released from the Cry4Ba-anti-Cry4Ba/Protein-A beads were separated by SDS-10% PAGE gel (Bio-Rad, Hercules, Calif.) and blotted to PVDF filter. The filter was blocked in 3% BSA-PBST for 1 h and then probed with α-V5 antibody (Invitrogen, Carlsbad, Calif.) for 2 h at room temperature. After washing, the filter was developed by ECL kit (GE Healthcare) and exposed to X-ray film.

Preparation of Cry4Ba toxin and α-Cry4Ba serum. A Cry4Ba mutant, Cry4BRA, was used in all experiments and will be referred to herein as Cry4Ba. The mutated Cry4Ba has a trypsin cleavage-site removed by the replacement of R203 with an A residue (Abdullah, M. A. et al. 2003. Appl. Environ. Microbiol. 69: 5343-5353). Production of Cry4Ba crystals and purification of Cry4Ba toxin were as described previously by those authors. Trypsin-digestion of Cry4Ba protoxin according to Abdullah et al. (2003. Appl. Environ. Microbiol. 69: 5343-53) produced a ˜66 kDa toxin mosquitocidal fragment. Antiserum against Cry4Ba toxin was prepared in New Zealand White rabbits at the Animal Resources Facility at the University of Georgia.

As a general matter, classification of B.t. Cry proteins is well-known in the art, and such nomenclature in this application is consistent with that unless specifically indicated to the contrary. For example, see Revision of the Nomenclature for the Bacillus thuringiensis Crystal Proteins; Crickmore et al. Microbiology and Molecular Biology Reviews (1998) Vol 62: 807-813. The boundaries set in that paper are approximately 95, 78, and 45% sequence identity. (Thus, all Cry4 toxins have at least 45% identity with each other. All Cry4B proteins have at least 78% identity with all other Cry4Bs, etc.) See also their website and the full list of toxins there (lifesci.sussex.ac.uk/home/Neil_Crickmore/Bt/). Other Cry toxins (beyond Cry4s) for controlling dipterans can be selected from that list and used according to the subject invention.

Other mutant Cry4Ba proteins disclosed in Abdullah et al. 2003 include 4BL3PAT and 4BL3GAV. The L3GAV and L3PAT can appear in subscripts.

Dot blots. S2 cells expressing An. gambiae cadherin were solubilized in PBS containing 1% CHAPS and Complete™ protease inhibitor (Roche Applied Science) and the sample tube rotated at room temperature for 1 hour. The soluble proteins were applied to a HiTrap Ni²⁺-chelating HP column (GE Healthcare) and eluted with imidazole. The partially purified proteins were separated by SDS-PAGE and transferred to a PVDF filter for western blotting. The partially purified proteins were also dotted onto a PVDF filter directly and probed with ¹²⁵I-Cry4Ba, or with ¹²⁵I-Cry4Ba plus unlabeled Cry4Ba (1000-fold). The toxin was labeled with Na¹²⁵I (GE Healthcare) as described previously (Hua, G. et al. 2004. Insect Biochem. Molec. Biol. 34, 193-202). The filters were exposed to X-ray film at −80° C. for autoradiography.

The truncated cadherin peptides, CR11-MPED and TM-Cyto were expressed in E. coli and purified as previously described (Chen, J. et al. 2007. Proc. Natl. Acad. Sci. U.S.A. 104: 13901-13906). Various amounts of purified peptides were dotted on PVDF filters and probed with ¹²⁵I labeled Cry4Ba, or with ¹²⁵I labeled Cry4Ba plus unlabeled Cry4Ba (1000-fold) and exposed to X-ray film as above.

Mosquito larva bioassay. Soluble Cry4Ba was mixed with purified CR11-MPED or TM-Cyto peptides in 1:100 (toxin:cadherin peptide) mass ratios in distilled water. A total of 10 4^{th} instar larvae per 2 ml of water with replicates in a 6-well Costar culture plate were fed soluble Cry4Ba toxin or a mixture of toxin plus cadherin peptide. Mortalities were scored after 16 h at 27° C. Bioassays were repeated three times for each treatment.

### EXAMPLE 2

### Results for Cloning and Analysis of An. Gambiae Cadherin AgCad1, Cry4Ba Binding to AgCad1 and AgCad1 Enhancement of Cry4Ba Toxicity to An. Gambiae Larvae

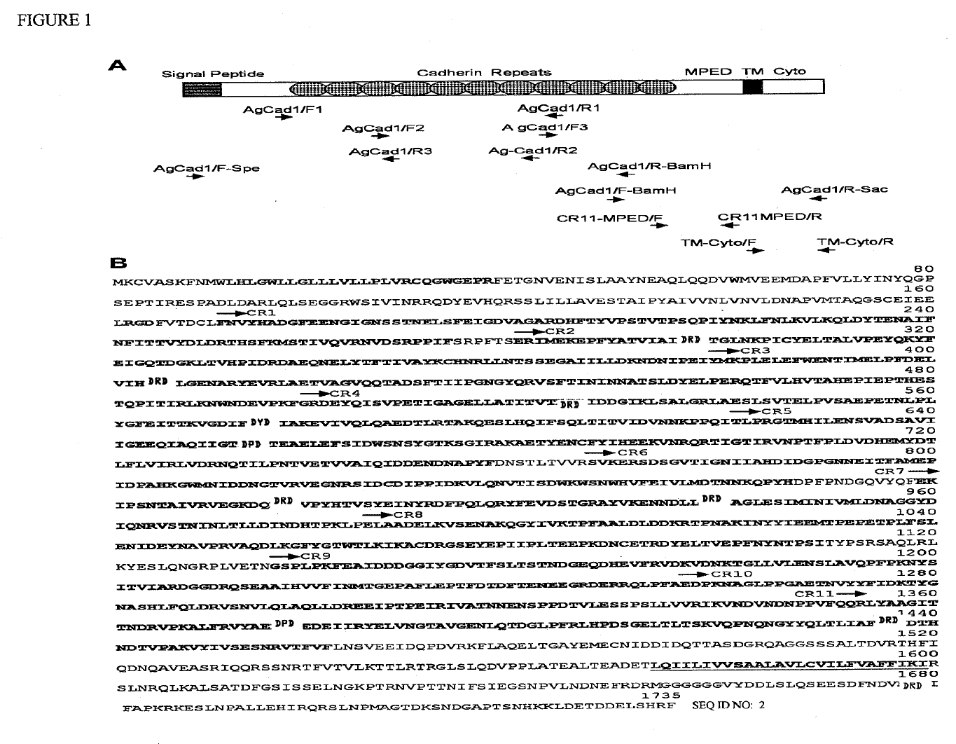

Cloning AgCad1. To identify cadherins in An. gambiae larvae that are similar to the lepidopteran cadherins located in the midgut brush border, we searched An. gambiae databases using BLAST with the Bt-R₁ cadherin from M. sexta. The predicted cadherin sequence (XM_{—}312086) was selected as the most homologous sequence. Using PCR and RACE as diagrammed in FIG. 1A, we cloned a cDNA corresponding to this An. gambiae cadherin (FIG. 1B). The coding sequence encodes a protein, designated AgCad1, of 1735 amino acids with a predicted molecular weight of 195 kDa. Sequence analysis identified a signal peptide at its N-terminus followed by 11 CR, a MPED and a TM domain. Ten putative calcium-binding sequences are distributed throughout the extracellular domain. Integrin recognition sequences, RGD (Pierschbacher, M. D. and Ruoslahti, E. 1984. Nature 309: 30-33) and LDV (Wagner, E. A. et al. 1989. J. Cell Biol. 109: 1321-1330; Tselepis, V. H. et al. J. Biol. Chem. 272: 21341-21348) are located before CR1 and in CR5, respectively. The cytoplasmic domain consists of 136 amino acids and a calcium-binding recognition sequence (DRD).

An. gambiae cadherin AgCad1 is expressed in midgut tissue. The presence of AgCad1 in the midgut of larvae was confirmed by PCR analysis and immunohistochemistry experiments. Gene-specific primers were designed to amplify a 636 bp fragment of AgCad1. PCR amplification using gut cDNA and plasmid pIZT-AgCad1 as templates with gene-specific primers resulted in the expected size products. As a control, PCR primers were designed to a second predicted An. gambiae cadherin-like protein; the protein in An. gambiae databases most homologous to AgCad1. The PCR product detected using PCAP-primers and gut cDNA was smaller in size than the AgCad1 PCR product and no product was detected with pIZT-AgCad1 as template. In summary, expression of AgCad1 in An. gambiae gut cDNA was detected by PCR.

To confirm the presence of AgCad1 protein in midgut tissue, we used α-AgCad1 to probe sectioned gut tissues. The serum is relatively specific for AgCad1 as no cross-reaction was detected to PCAP in western blot experiments. Immunostaining localized cadherin to the microvilli in the posterior midgut. The control sections probed with pre-immune serum and secondary labeled antibody showed only faint background staining. We conclude from gene-specific PCR and immunolocalization experiments that AgCad1 is expressed in midgut tissue, and the protein is localized on the brush border membrane.

More particularly, localization of cadherin-like protein was in the posterior region of the midgut of larval A. gambiae. AgCad1 was immunostained on the microvilli. As a control, midgut sections probed with pre-immune sera were not immunostained. Rhodamine-labeled Cry4Ba localized on microvilli of posterior midgut, but labeled BSA did not bind to any part of the midgut (microvilli MV, basal lamina BL) (Bars 50 μm).

Cry4 toxins bind the apical brush border of midgut cells in the gastric caecae and posterior gut of An. gambiae (Ravoahangimalala, O. and Charles, J. F. 1995. FEBS Lett. 362: 111-115). We probed sectioned gut tissue of An. gambiae with rhodamine-labeled Cry4Ba; Cry4Ba bound to midgut microvilli in the posterior midgut, in a pattern similar to AgCad1 localization. A rhodamine labeled-BSA control showed faint non-specific binding to gut tissue.

Anti-AgCad1 serum and Cry4Ba detect a 200 kDa protein in An. gambiae BBMV. The molecular size of AgCad1 in brush border membrane was determined to be about 200 kDa by probing blots of BBMV proteins with anti-AgCad1 serum. This size is slightly larger than the 195 kDa predicted size suggesting post-translational modification, most likely by glycosylation. The α-AgCad1 serum also detected a 25 kDa peptide that may be a degraded form of AgCad1 or a cross-reactive protein. When strips from the same blot of An. gambiae BBMV proteins were probed with Cry4Ba toxin, a 200 kDa protein was detected. Proteins of 80 kDa and 28 kDa were also detected by Cry4Ba toxin.

AgCad1 expressed in S2 cells binds Cry4Ba. Transient expression of AgCad1 in S2 cells provided alternate approaches to test for Cry4Ba to binding to AgCad1. S2 cells were transfected with pIZT or pIZT-AgCad1 and probed with either α-AgCad1 serum or Cry4Ba toxin. A 200 kDa AgCad1 was expressed in pIZT-AgCad1 transfected cells. Although Cry4Ba bound many S2 cell proteins, no Cry4Ba binding was detected to expressed AgCad1 protein. We considered that AgCad1 expressed by S2 cells was different than AgCad1 expressed on midgut brush border and not detected under denaturing conditions. To facilitate measuring toxin binding under non-denaturing conditions AgCad1 was partially purified from pIZT-AgCad1-transfected S2 cells using a Ni-affinity column (FIG. 2A). The eluted cadherin fraction was dotted in increasing amounts to a membrane filter and then the filter probed with ¹²⁵I-Cry4Ba (FIG. 2B). As the amount of dotted protein increased, more ¹²⁵I-Cry4Ba was bound and excess unlabeled Cry4Ba (1000-fold) competed ¹²⁵I-Cry4Ba binding (FIG. 2B). Since Cry4Ba displayed non-specific binding to S2 cells proteins on blots, proteins from pIZT vector-transfected cells were applied to a Ni-affinity column and eluted and tested for Cry4Ba binding. This was possible due to non-specific binding of S2 cell proteins to the Ni-affinity column (data not shown). While a strong signal was detected for ¹²⁵I-Cry4Ba binding to partially purified AgCad1 protein, a weak signal was detected for dotted S2 cell protein (FIG. 2C). The ability of Cry4Ba, but not Cry1Ab (a lepidopteran-active toxin) to compete ¹²⁵I-Cry4Ba binding (FIG. 2C) was further evidence that binding was specific.

Bead extraction of AgCad1 expressed on Dm S2 cells. Bead extraction experiments provided a second approach for testing Cry4Ba binding to cadherin expressed in S2 cells. Cry4Ba was coupled indirectly to Protein A beads via an α-Cry4Ba antibody (Experimental Methods) and the bead complex was incubated with S2 cells expressing AgCad1. Extracted proteins were separated by SDS-PAGE, blotted to a membrane filter, and AgCad1 detected with α-V5 mouse antibody. Using α-V5 mouse antibody to detect the C-terminal V5 epitope tag on expressed AgCad1, circumvented detection of rabbit antibodies attached to the Cry4Ba-bead complex. AgCad1 was detected in S2 cells, primarily as a mixture of 200-, 55-, and 29-kDa bands. The Cry4Ba-bead complex extracted the three AgCad1 peptides and as more Cry4Ba was added to the bead complex, more AgCad1 was extracted. Some AgCad1 was extracted by the bead-protein A and the beads-protein A-α-Cry4Ba. The extracted 50 kDa protein correlated with the presence of antibody on the beads, but not Cry4Ba toxin. Since the 55 and 29 kDa peptides bound Cry4Ba and were detected by α-V5 antibody, those fragments probably correspond to C-terminal fragments of AgCad1. The Cry4Ba extraction experiments are further evidence that Cry4Ba binds AgCad1 and suggest that Cry4Ba binds at the C-terminal region of AgCad1. A binding site near the C-terminus is consistent with the current model for Cry1A toxin binding to lepidopteran cadherins, where the Cry1A toxins bind the cadherin repeat nearest the C-terminus of the protein (Dorsch, J. A. et al., 2002. Insect Biochem. Mol. Biol. 32: 1025-1036; Xie, R. et al. 2005. J. Biol. Chem. 280: 8416-8425; Hua, G. et al. 2004. J. Biol. Chem. 279: 28051-28056).

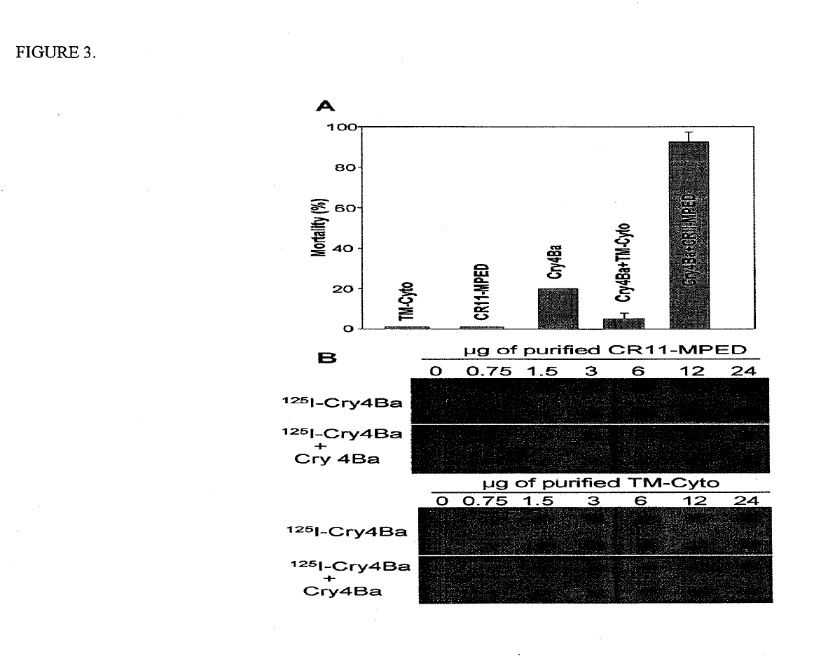

The CR11-MPED region of AgCad1 enhances Cry4Ba toxicity to An. gambiae larvae and binds Cry4Ba toxin. We expressed the CR11-MPED region from AgCad1 in E. coli and tested the purified 24-kDa peptide with Cry4Ba in bioassays of mosquito larvae. As shown in FIG. 3, the CR11-MPED peptide increased the toxicity of Cry4Ba against An. gambiae 4^{th} instar larvae. Cry4Ba alone at 0.25 μg/ml killed 20% of the larvae, whereas Cry4Ba:CR11-MPED (1:100 mass ratio) increased mortality to 92.5%. The peptide alone was not toxic to A. gambiae larvae. A similar-sized peptide comprised of the TM-Cyto region reduced Cry4Ba toxicity to only 5% larval mortality.

Since CR11-MPED enhanced Cry4Ba toxicity, and toxin binding was required for CR12-MPED enhancement of Cry1A toxicity (Chen et al. 2007. Proc. Natl. Acad. Sci. U.S.A. 104: 13901-13906), Cry4Ba was tested for the ability to bind CR11-MPED from AgCad1. Increasing amounts of the truncated AgCad1 peptides (CR11-MPED or TM-cyto) were dotted onto a membrane filter and the filter probed with ¹²⁵I-labeled Cry4Ba. Binding was visualized through autoradiography to X-ray film. Labeled Cry4Ba bound to CR11-MPED stronger than to TM-Cyto on dot blots, though both bound at low levels (FIG. 3). Excess unlabeled Cry4Ba toxin (1000-fold) partially reduced the binding to CR11-MPED but had no effect on binding to TM-Cyto peptide.

### EXAMPLE 3

### Materials and Methods for Partial Cadherin Fragments from Anopheles Gambiae AgCad1 Synergize Cry4Ba Toxicity Against Aedes Aegypti Larvae

Mosquito rearing. A. aegypti was maintained at 27±1° C., 65% relative humidity with a photoperiod of 14 h light: 10 h dark. Adults were fed with 10% sucrose solution and females were blood-fed on mice for 30 min or until engorged. Laid eggs were collected and hatched in distilled water. Larvae were fed ground fish food (TetraMin) daily and grown until 4^{th} instar for bioassay.

Preparation of Cry4Ba. A Cry4Ba mutant, Cry4BRA, was used in all experiments and will be referred to herein as Cry4Ba. The mutated Cry4Ba has a trypsin cleavage-site removed by the replacement of R203 with an A residue (Abdullah, M. A. et al. 2003. Appl. Environ. Microbiol. 69: 5343-53; Angsuthanasombat, C., N. et al. 1993. FEMS Microbiol Lett 111: 255-6). Production and purification of Cry4Ba inclusion bodies and trypsin-activated Cry4Ba toxin were as described previously (Abdullah, M. A. et al. 2003. Appl. Environ. Microbiol. 69: 5343-53). Cry4Ba inclusion body form (IBF) protoxin was suspended in sterilized deionized water, while the purified soluble form (SF) of trypsin-activated toxin was purified and finally dialysed against deionized water.

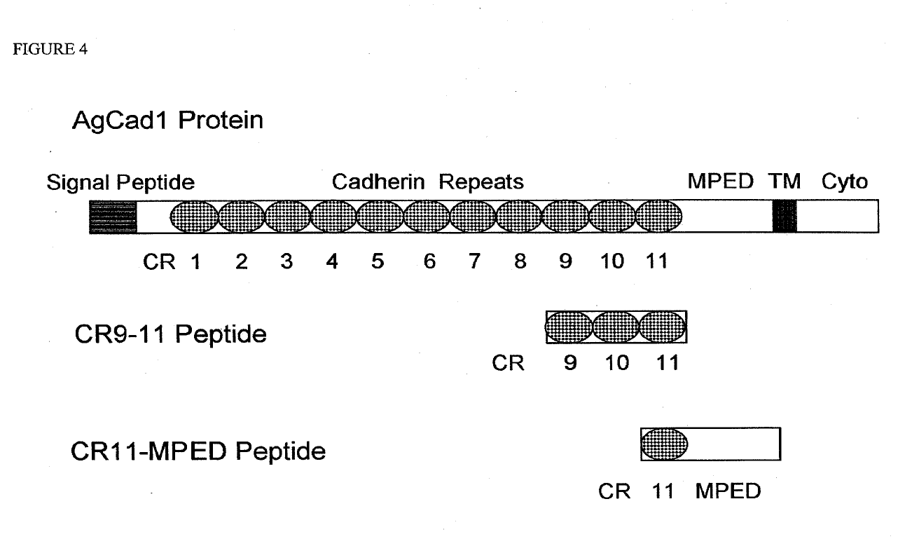

An. gambiae cadherin fragment construction and purification. The truncated cadherin fragment (CR9-11; shown in FIG. 4) was constructed from AgCad1. The CR9-11 region was amplified by PCR using the plasmid pMECA-AgCad1-3′ (Hua et al., 2008. Biochemistry, 47:5101-5110) as a template with a pair of primers, 5′-GAC CCA TAT GGA CGA AAC GCT GCA CAT CCT GA-3′ (SEQ ID NO:28) and 5′-ACA CCT CGA GGA ACC GGT GGG ACA GCT CGT CGT CA-3′ (SEQ ID NO:29) for CR11-MPED, and 5′-CGA GCA TAT GGG GTC CCC G TT GCC GAA ATT (SEQ ID NO:32) and 5′-CGC TCT CGA GAA ACA C GA ACG TCA CGC GGT TC (SEQ ID NO:33) for CR9-11. The amplified PCR fragment was cleaved with Nde I and Xho I, and then cloned into the pET-30a(+) vector (Novagen, Madison, Wis.) named pET-AgCad1/CR9-11, and the construct was transformed into E. coli strain BL21-CodonPlus (DE3)/pRIL (Stratagene, LaJolla, Calif.). The cloned cadherin fragment was confirmed by DNA sequencing in both forward and reverse directions at the Molecular Genetics Instrumentation Facility at University of Georgia. The CR11-MPED region (shown in FIG. 4; Hua et al., 2008, Biochemistry. 47:5101-5110; SEQ ID NOs: 3 and 4) and the CR9-11 (SEQ ID NOs: 5 and 6) region were over-expressed by induction with 1 mM isopropyl β-D-thiogalactopyranoside (IPTG) when the culture OD₆₀₀ reached 0.5-0.6. The expression and purification protocols for the truncated cadherin fragments were as described in a previous paper (Chen, J. et al. 2007. Proc. Natl. Acad. Sci. U.S.A. 104:13901-13906. The inclusion body form (IBF) was prepared as a suspension in sterilized deionized water, while the purified solubilized form (SF) of both CR9-11 and CR11-MPED was dialysed against distilled water. Total protein amount was measured by Bio-Rad protein assay using bovine serum albumin (BSA) as standard (Bradford, M. 1976. Anal. Biochem. 72: 248-254). One microgram of each cadherin peptide was analyzed by sodium dodecyl sulfate-15% polyacrylamide gel electrophoresis (SDS-15% PAGE) with Coomassie brilliant blue R-250 staining. Specific concentration of target protein, such as toxin or the cadherin peptide in total protein was determined from Coomassie-stained gel by gel image analyzer (Alpha Innotech, San Leandro, Calif.) using BSA as standard.

Mosquito larva bioassay. Fragments CR9-11 and CR11-MPED were tested in this experiment for analysis of synergistic mosquito-larvicidal effect of partial cadherin fragments with Cry4Ba toxin on A. aegypti larvae.

Bioassays were set as described below:

Part I: To determine the LC₅₀ value of the IBF Cry4Ba: 4^{th} instar larvae were treated with 0, 1, 2, 4, 8, 16, 32, and 64 ng/ml Cry4Ba using a 6-well Costar culture plate with 2 ml of distilled H₂O in each well.

Part II: To test the synergistic effect, IBF (12.5 ng/ml) of Cry4Ba was mixed with IBF of CR9-11 or CR11-MPED at different toxin:peptide mass ratios (1:0, 1:1, 1:2, 1:5, 1:10, 1:25, 1:50, and 1:100). The partial cadherin-like protein from western corn rootworm (WCRW) Diabrotica virgifera virgifera, WCR8-10, was used as negative control. The cloning, expression, and purification of the WCR8-10 was described in Park et al., 2009 (Appl. Environ. Microbiol. March 27. epub ahead of print). Inclusion body form of WCR8-10 was finally prepared as a suspension in sterile deionized water. IBF (12.5 ng/ml) of Cry4Ba was mixed with IBF of WCR8-10 at 1:100 mass ratio.

Part III: To determine the shift in LC₅₀ of Cry4Ba due to either CR9-11 or CR11-MPED, a fixed mass ratio of toxin:peptide of 1:25 was used with the same range of toxin concentrations used in part I above. The 1:25 ratio was chosen based on the bioassay result in part II above that shows that the synergistic effect was at the maximum and also began to saturate at this ratio (no significant difference was observed at higher ratio). The relative toxicity defined as the ratio of the lethal concentration of Cry4Ba alone to the lethal concentration of Cry4Ba with the cadherin peptides, was determined for each of the combinations. When the ratio was greater than 1, the protein interaction was considered synergistic because toxicity exceeded the value expected from the individual additive toxicity. When the ratio was less than 1, the interaction was considered antagonistic, whereas a ratio of 1 indicated that there was no effect. The cadherin peptides by themselves caused no observable toxicity to the mosquito larvae.

Each treatment was replicated 4 times and each replicate contained 10 larvae. Larval mortality was recorded after 16 hr.

### EXAMPLE 4

### Results—CR11-MPED and CR9-11 of AgCad1 Synergize Cry4Ba Toxicity to Yellow Fever Mosquito, Aedes Aegypti

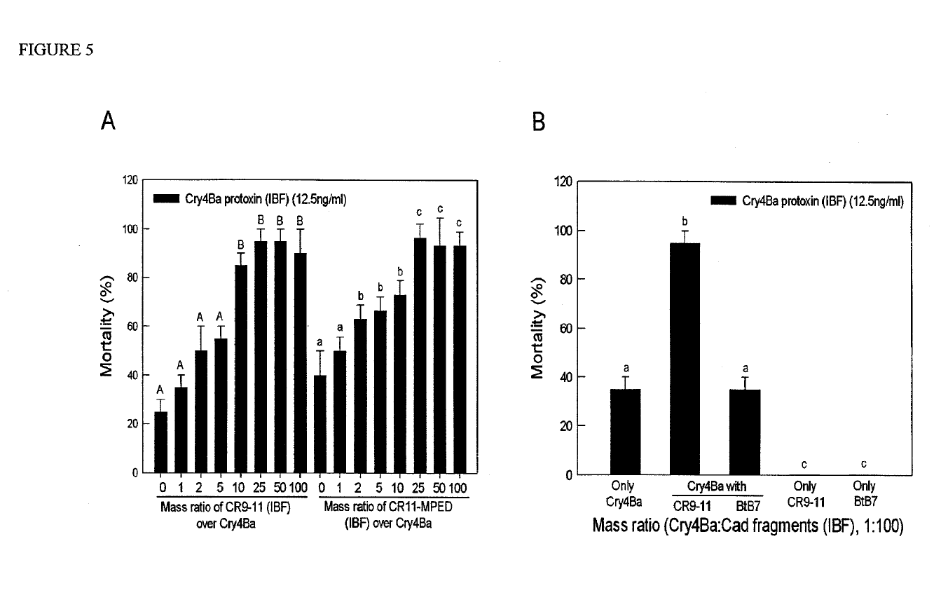

Feeding Cry4Ba alone or Cry4Ba with CR9-11 (IBF or SF) or CR11-MPED (IBF or SF) to A. aegypti larvae showed enhanced toxicity when the partial AgCad1 fragments were present. The calculated LC₅₀ mortality value of Cry4Ba IBF was 20.34 (16.37-25.93) ng/ml (Table 1; FIGS. 5A and 5B). The addition of IBF of CR9-11 and CR11-MPED at a 1:25 mass ratio of Cry4Ba:cadherin fragment peptide to the crystal suspensions not only reduced the Cry4Ba LC₅₀ values, 3.43 (1.66-5.80) and 7.35 (5.94-9.07) ng/ml, respectively (Table 1; FIG. 4A), but SF of CR9-11 and CR11-MPED also reduced the Cry4Ba LC₅₀ values, 5.79 (4.42-6.73) and 9.23 (7.53-11.33) ng/ml, respectively (Table 1; FIG. 5B).

The use of soluble form (SF) led to lower level of enhancement when compared to its inclusion body form (IBF) of the cadherin peptides. This might be explained by the fact that mosquito larvae are filter feeders, thus more peptides are ingested if they can be filtered by the mosquito. Schnell et al. suggested that mosquito larvae selectively concentrate particles while excluding water and soluble molecules, and reported that solubilized crystals of Bti were 7000 times less toxic to A. aegypti larvae than intact crystals (Schnell et al. 1984. Science. 223(4641): 1191-1193.

TABLE 1
Toxicity of Cry4Ba protoxin inclusion body alone
and with combinations of A. gambiae
cadherin fragments on 4^{th} instar larvae of A. aegypti

| | | LC₅₀ | | | Relative |
|---|---|---|---|---|---|
| Treatments | n | (95% CL)^{a} | Slope ± SE | χ² | Toxicity^{b} |
| Cry4Ba (IBF)^{c} | 280 | 20.34 | 2.03 ± 0.22 | 1.87 | — |
| | | (16.37-25.93) | | | |
| Cry4Ba (IBF) + | 280 | 7.35 | 2.05 ± 0.19 | 1.80 | 2.76 |
| CR11-MPED | | (5.94-9.07) | | | |
| (IBF) (1:25) | | | | | |
| Cry4Ba (IBF) + | 280 | 9.23 | 2.17 ± 0.21 | 1.91 | 2.20 |
| CR11-MPED | | (7.53-11.33) | | | |
| (SF)^{d} (1:25) | | | | | |
| Cry4Ba (IBF) + | 280 | 3.43 | 1.83 ± 0.34 | 2.18 | 5.93 |
| CR9-11 | | (1.66-5.80) | | | |
| (IBF) (1:25) | | | | | |
| Cry4Ba (IBF) + | 280 | 5.79 | 1.96 ± 0.21 | 2.46 | 3.51 |
| CR9-11 | | (4.42-6.73) | | | |
| (SF) (1:25) | | | | | |

^{a}Results are 50% lethal doses (LC₅₀) (with 95% confidence limits) and are expressed as nanograms of Cry protein per ml for bioassays. LC₅₀ values were calculated using EPA Probit Analysis Program Version 1.5. and the differences of LC₅₀ values are considered significantly different if the confidence limits do not overlap.
^{b}Relative toxicity was determined by dividing the LC₅₀ value of a Cry4Ba protoxin inclusion body alone with the LC₅₀ value of Cry4Ba protoxin inclusion body with each A. gambiae cadherin fragments.

In another bioassay using either SF or IBF forms of Cry4Ba and CR11-MPED (SF or IBF), we showed that the cadherin peptide could enhance both the protoxin and toxin forms of Cry4Ba (FIG. 4). For the IBF samples, maximum level of enhancement was obtained at 1:25 (Cry4Ba:CR11-MPED) mass ratio. However, for the SF samples, higher protein amounts and higher mass ratio was needed to achieve the same level of mortality compared to the IBF samples. To obtain a better comparison of the enhancement effect of CR11-MPED and CR9-11, a bioassay was done using only the IBF samples. Dose-toxicity bioassays showed that CR9-11 (IBF) was significantly better than CR11-MPED (IBF) in enhancing Cry4Ba against A. aegypti larvae (FIG. 5, Table 1).

### EXAMPLE 5

### Materials and Method. AgCad CR11-MPED, AgPCAP CR11-MPED, and MsCad CR12-MPED Synergize Cry4Ba Toxicity to An. Gambiae Larvae

Cloning a cDNA encoding the An gambiae cadherin-like protein called AgPCAP (AJ439060; Ano-PCAP). Cadherins from Lepidoptera and Diptera with similarity to Bt-R1 were selected from protein databases using BLASTP and a 969 amino acid fragment of Bt-R1. The set of selected proteins included cadherins from M. sexta, Bombyx mori, Lymantria dispar, Ostrinia nubilalis, Heliothis virescens and Pectinophora gossypiella that have evidence for function as Cry receptors. The BLASTP search also retrieved predicted expressed peptides for cadherin-like proteins from Drosophila melanogaster and An. gambiae. The An. gambiae cadherin-like protein AgCad1, deduced from a predicted expressed sequence tag (AAAB01008859) is 968 amino acid residues in length with 34% residue identity and putative cell adhesion protein (AJ439060; Ano-PCAP) consists of 1881 residues that have 24% amino acid identity with Bt-R1 cadherin.

Using the DNA sequences encoding partial proteins we cloned a cDNA fragment corresponding to the peptide and then the full-length cDNA as follows. Total mRNA was isolated from An. gambiae larvae, cDNA prepared and then a fragment of the coding region amplified using primers designed from database sequences. The Anopheles cDNA fragments were cloned, sequenced and then extended using 5′ and 3′ RACE methodology as described in Hua et al. (2004, Insect Biochem Molec. Biol.). The full-length nucleotide and amino acid sequence for AgPCAP is presented as SEQ ID NOs: 7 and 8.

A partial cadherin peptide corresponding to CR11-MPED from AgPCAP was made. The CR9-11 region was amplified by PCR using the plasmid pMECA-AgCad1-3′ (Hua et al, Biochemistry, in press,) as a template with a pair of primers, Ag-PCAP/CR11-F:TTCAccatgGGTATCTCAACGTCGTCGCTGTTCGG (SEQ ID NO:34) and An-PCAP/MPED-R:CATACTCGAGTGACGGACAGCTCGTCCATCTCTGC (SEQ ID NO:35). Amplified PCR fragment was cleaved with Nde I and Xho I, and then cloned into the pET-30a(+) vector (Novagen, Madison, Wis.) named pET-AgCad1/CR9-11, and the construct was transformed into E. coli strain BL21-CodonPlus (DE3)/pRIL (Stratagene, LaJolla, Calif.). The cloned cadherin fragment was confirmed by DNA sequencing in both forward and reverse directions at the Molecular Genetics Instrumentation Facility at University of Georgia.

Cloning of the CR12-MPED region of Bt-R1a. Cloning of the cadherin Bt-R₁ₐ (GenBank AY094541) from M. sexta larvae has been described by Hua et al. (Insect Biochem Molec Biol 2004. 34, 193-202). The nucleotide and amino acid sequence for full-length Bt-R1a are presented in SEQ ID NOs: 11 and 12. The cDNA encoding Bt-R₁ cloned in the pIZT vector (Invitrogen Co., Carlsbad, Calif.) was used as template for subcloning the CR12-MPED fragment (amino acids G1362 to P1567) by PCR with primers: 5′-GTACCATATGGGGATATCCACAGCGGACTCCATCG-3′ (SEQ ID NO:36) and 5′-GGCTCTCGAGAGGCGCCGAGTCCGGGCTGGAGTTG-3′ (SEQ ID NO:37). The resulting PCR fragments were gel purified, digested by Nde I and Xho I endonucleases, and then subcloned into the pET-30a (+) vector (Novagen, Inc., Madison, Wis.) to yield plasmids pET-CR12-MPED. The coding sequences and clone orientation were confirmed by sequencing. The nucleotide and amino acid sequences for CR12-MPED are shown in SEQ ID NOs: 13 and 14.

Production of cadherin peptides in E. coli. The pET-constructs were transformed into E. coli strain BL21(DE3)/pRIL (Stratagene Co., La Jolla, Calif.), and positive clones were selected on LB plates containing kanamycin and chloramphenicol. The CR peptides were over-expressed in E. coli as inclusion bodies. Inclusion bodies were solubilized and proteins purified on a HiTrap™ Ni²⁺-chelating HP column (GE Healthcare, Piscataway, N.J.). Purified proteins were dialyzed against 10 mM Tris-HCl, pH 8.0) at 4° C. Protein concentration was quantified by the method of Bradford with bovine serum albumin (BSA) as standard. Purified CR12-MPED was stored at −20° C.

Preparation of Cry4Ba toxin. A Cry4Ba mutant, Cry4BRA, was used in all experiments and will be referred to herein as Cry4Ba. The mutated Cry4Ba has a trypsin cleavage-site removed by the replacement of R203 with an A residue. Production of Cry4Ba crystals and purification of Cry4Ba toxin were as described previously (Abdullah et al. 2003. Appl. Environ. Microbiol. 69: 5343-53). Trypsin-digestion of Cry4Ba protoxin according to Abdullah et al. (2003. Appl. Environ. Microbiol. 69: 5343-53) produced a ˜66 kDa toxin mosquitocidal fragment.

Preparation of Cry11BA. The Cry11BA protein was prepared from Bt strain 407, harboring plasmid pJEG80.1 encoding cry11Ba (Delecluse et al. Appl Environ Microbiol 1995, 61, 4230-4235). A complex sporulation medium supplemented with erythromycin antibiotic was prepared: 2 gm/liter peptone (Difco), 5 gm/liter yeast extract (Difco), 0.07 M K2HPO4, 0.02 M KH2PO4, 6×10−3 M glucose, 2×10−4 M MgSO4.7H2O, 5×10−4 M CaCl2.2H2O, 6×10−6 M MnSO4.7H2O, 1×10−6 M FeSO4.7H2O. The culture was shaken in 1 liter medium in a 4 liter flask at 30° C. overnight, then 1 liter of sodium phosphate solution (0.06 M Na2HPO4, 0.04 M NaH2PO4.H2O) was added into the growing culture. After sporulation, spores and crystals were harvested by centrifugation and then re-suspended in 0.1 M NaCl, 2% Triton-X 100, 20 mM Bis-Tris (pH 6.5). The suspension was sonicated on ice, and then the spore crystal mixture was washed in the 0.1 M NaCl, 2% Triton-X 100, 20 mM Bis-Tris (pH 6.5) three times, 1 M NaCl (twice), and distilled H₂O (twice). All centrifugation steps were 10,000×g, 10 min 5° C. Crystals were separated from spores by centrifugation through a 30-60% (w/v) NaBr step gradient at 47,000×g for 2 h at 5° C. Purified crystals were washed twice with distilled water, dissolved in 20 mM NaOH at 37° C. for 2 h and dialyzed against 20 mM Na2CO3, 0.3 M NaCl, pH 9.6.

Mosquito larva bioassay. Soluble Cry4Ba was mixed with purified CR11-MPED or TM-Cyto peptides in 1:100 (toxin:cadherin peptide) mass ratios in distilled water. A total of 10 4^{th} instar larvae per 2 ml of water with replicates in a 6-well Costar culture plate were fed soluble Cry4Ba toxin or a mixture of toxin plus cadherin peptide. Mortalities were scored after 16 h at 27° C.

### EXAMPLE 6

### Results. Cry Toxin Enhancing Properties of AgPCAP CR11-MPED, AgCad CR11-MPED, and MsCad (Bt-R₁ₐ) CR12-MPED

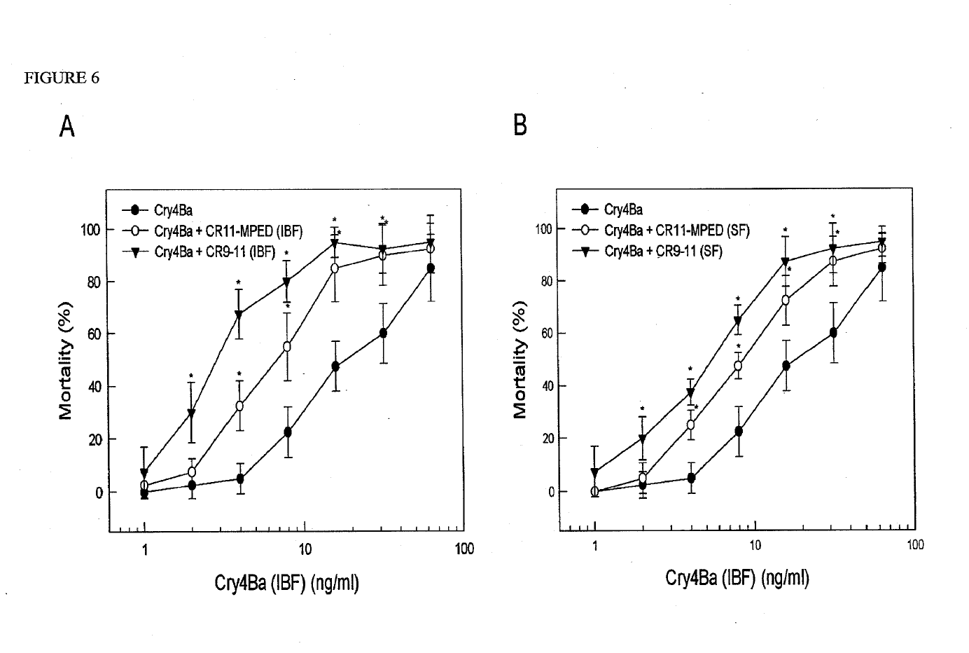

Bioassays established the dose-response for Cry4Ba toxin (SF) against 4^{th} instar larvae of An. gambiae. Using the results of this dose response to Cry4Ba bioassays with mosquito larvae were done with purified inclusion body forms (IBF) of AgCad1 CR11-MPED, AgPCAP CR11-MPED and MsCad (Bt-R1A CR12-MPED) and soluble Cry4BRA toxin (note Cry4BRA is a protease stable version of Cry4Ba described previously used to facilitate toxin purification). In FIG. 6, we showed that CR11-MPED of AgCad1 or AgPCAP and CR12-MPED of MsCad was able to significantly enhance Cry4Ba against the important human disease vector, An gambiae. The result that a cadherin peptide from a lepidopteran insect can enhance the dipteran-active toxin is unexpected.

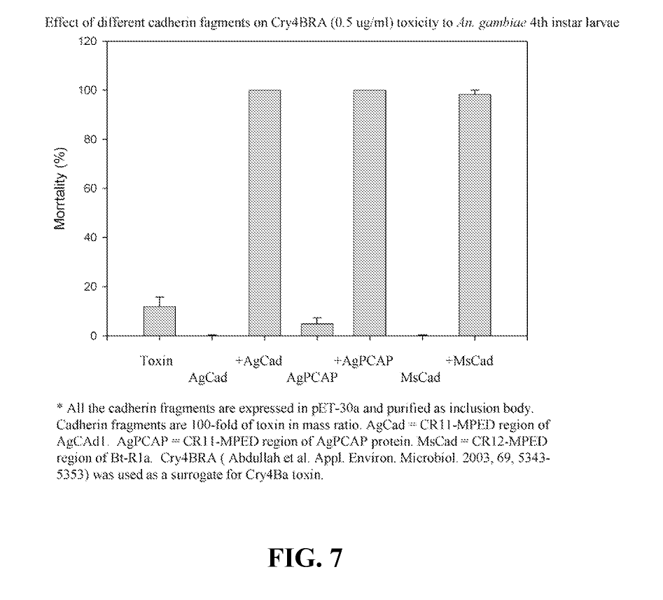

The mortality of 4th instar An. gambiae larvae caused by ingestion of various doses of Cry11Ba toxin (soluble form) is presented in FIG. 7. Larval mortality was scored 16 h after treatment. Using the results of this dose response to Cry11Ba Bioassays with mosquito larvae were done with purified inclusion body forms (IBF) of AgCad1, AgPCAP and MsCad and soluble Cry11Ba toxin. We showed that only CR11-MPED of AgPCAP and not CR11-MPED of AgCad or CR12-MPED of MsCad was able to significantly enhance Cry11Ba against the important human disease vector, An gambiae. These results demonstrated a specificity of cadherin fragments that enhance Cry11Ba toxicity to a mosquito larvae.

### EXAMPLE 7

### Materials and Methods. Binding Affinities of Cry4Ba Toxins to AgCad CR9-11 and AgCad CR11-MPED

To determine the binding affinities of Cry4Ba, a protein-protein binding assay using coated microtiter plates and enzyme-linked immunosorbent assay (ELISA) was performed according to Park et al. (2009. Apple Environ. Microbiol. Published on-line Mar. 27, 2009. The AgCad peptides were purified from inclusion bodies on a HiTrap Ni²⁺-chelating HP column (GE Healthcare, Piscataway, N.J.) according to Chen et al. (2007. Proc. Natl. Acad. Sci. U.S.A. 104:13901-13906). Purified CR8-10 peptide was dialyzed against PBS at 4° C. and quantified by the dye-binding method of (BioRad; Richmond, Calif.) with BSA as standard. Purified AgCad peptides were biotinylated using a 50-fold molar excess of sulfo-NHS-PC-biotin according to the manufacturer's (Pierce, Rockford, Ill.) instructions. The final reaction was dialyzed against 200 mM NaCl, 20 mM Na₂CO₃, pH 8.0 at 4° C. and stored in aliquots at 4° C. until needed for binding assays. Cry4Ba toxin were prepared from crystals and E. coli-derived inclusion bodies, respectively, using chymotrypsin in a previously described method (26). Microtiter plates (high binding 96-well, Immulon® 2HB, Thermo Fisher Scientific Inc., Waltham, Mass.) were coated with 1.3 μg Cry3Aa/well or 0.5 μg Cry3Bb/well in 50 μl coating buffer (100 mM Na₂CO₃, pH 9.6). Toxin coated plates were washed with wash buffer (PBS plus 0.05% Tween 20), blocked with 0.5% BSA in wash buffer, and incubated for 2 h with increasing concentrations of biotinylated CR8-10 peptide (0.01 nM to 18 nM) to determine total binding. Non-specific binding was determined by incubating the plates with increasing concentrations of biotinylated AgCad peptide with 1000-fold molar excess of non-labeled homologous AgCad peptide. Plates were washed, incubated with horseradish peroxidase conjugated streptavidin (SA-HRP; Pierce) diluted 1:10,000 in wash buffer, washed, and incubated with HRP chromogenic substrate (1-Step™ Ultra TMB-ELISA, Thermo Fisher Scientific Inc.) to detect bound SA-HRP. Color development was stopped by adding 3M sulfuric acid and absorbance was measured at 450 nm using a microplate reader (MDS Analytical Technologies, Sunnyvale, Calif.). Specific binding was determined by subtracting non-specific binding from total binding. Data were analyzed using SigmaPlot software (Version 9; Systat Software Inc., San Jose, Calif.) and the curves were fitted based on a best fit of the data to a one site saturation binding equation.

### Results (Example 7)—Binding Affinities of Cry4Ba Toxins to AgCad CR9-11 and AgCad CR11-MPED

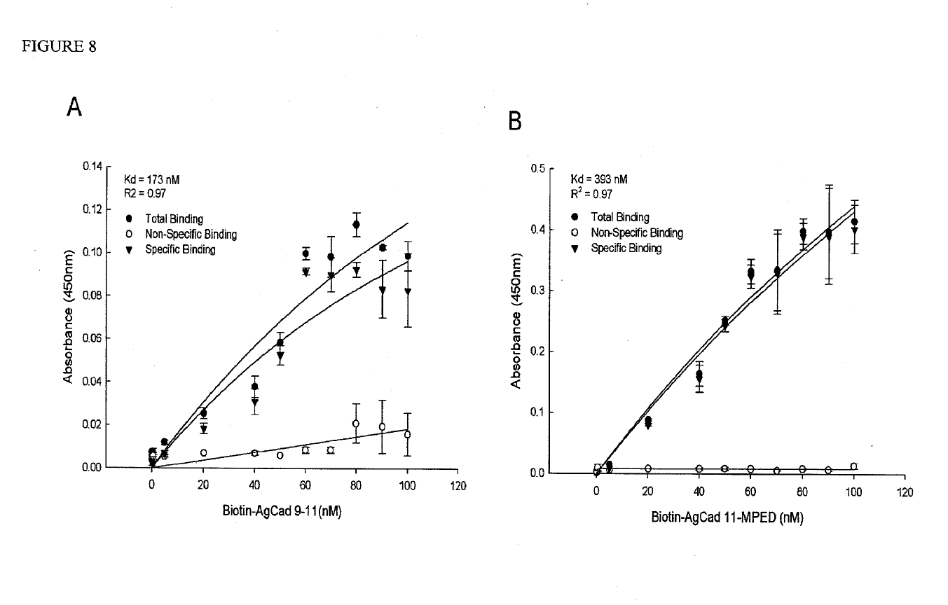

The AgCad1 domains are typical of an insect midgut cadherin. AgCad1 is located in the apical brush border of the posterior midgut of 4^{th} instar An. gambiae larvae. Cry4Ba binds both AgCad fragments (FIG. 8). AgCad-CR9-11 and AgCad-CR11-MPED fragment bind Cry4Ba toxin relatively low affinity (173 nm and 393 nM, respectively), about 50- to 100-fold lower than the affinity of Cry1Ab to the terminal CR12 cadherin domain of Bt-R₁ (Chen et al. 2007. Proc. Natl. Acad. Sci. U.S.A. 104:13901-13906). Cry4Ba binds within the region of AgCad1 that includes the C-most CR repeat (CR11) and the MPED region (FIG. 8, Panel B).

1. A method of inhibiting a dipteran, said method comprising providing a polypeptide to said dipteran insect for ingestion, wherein said polypeptide binds a Bacillus Cry protein, wherein said polypeptide comprises at least 75 amino acid residues, and said polypeptide is at least 85% identical with at least 75 contiguous amino acid residues of a midgut cadherin ectodomain from an insect, wherein said cadherin is a Bacillus Cry binding protein.

2. The method of claim 1, wherein said polypeptide comprises at least 100 amino acids and is at least 85% identical with at least 100 contiguous amino acid residues of said cadherin.

3. The method of claim 1, wherein said polypeptide is at least 90% identical with at least 100 contiguous amino acid residues of said cadherin.

4. The method of claim 1, wherein said polypeptide comprises at least 100 amino acids and is at least 95% identical with at least 100 contiguous amino acid residues of said cadherin.

5. The method of claim 1, said method further comprising providing a Bacillus Cry protein to said dipteran for ingestion.

6. The method of claim 5, wherein said Bacillus is of a species selected from the group consisting of thuringiensis and sphaericus.

7. The method of claim 5, wherein said Cry protein is a Cry4 protein.

8. The method of claim 5, wherein said Cry protein is selected from the group consisting of a Cry4A protein and a Cry4B protein.

9. The method of claim 5, wherein said Cry protein is a Cry4Ba protein.

10. The method of claim 1, wherein said dipteran is a larvae.

11. The method of claim 1, wherein said dipteran is a mosquito.

12. The method of claim 11, wherein said mosquito is of a genus selected from the group consisting of Anopheles, Aedes, and Culex.

13. The method of claim 1, wherein said dipteran is a Nematoceran.

14. The method of claim 1, wherein said dipteran is a fly.

15. The method of claim 14, wherein said fly is a black fly.

16. The method of claim 1, wherein said dipteran is selected from the group consisting of fungus gnats, sand flies, and midges.

17. The method of claim 1, wherein said dipteran is of a genus selected from the group consisting of Simulium, Orefelia, Phlkebotomus, Sergentomyia, Lutzomya, Tipula, and Chironimus.

18. The method of claim 1, where said cadherin is from a dipteran insect.

19. The method of claim 1, where said cadherin is from a mosquito.

20. The method of claim 19, where said mosquito is of a genus selected from the group consisting of Anopheles and Aedes.

21. The method of claim 1, where said cadherin is selected from the group consisting of AgCad or AgPCAP

22. The method of claim 1, where said polypeptide comprises an amino acid sequence selected from the group consisting of SEQ ID NO:4 and SEQ ID NO:10 (CR11-MPEDs)

23. The method of claim 1, where said polypeptide comprises SEQ ID NO:6 (CR9-11).

24. The method of claim 1, where said polypeptide comprises CR11.

25. The method of claim 1, where said cadherin is from a lepidopteran insect.

26. The method of claim 1, where said cadherin is BtR1.

27. The method of claim 1, where said polypeptide comprises an amino acid sequence selected from the group consisting of SEQ ID NO:14 (CR12-MPED) and CR12.

28. The method of claim 1, where said polypeptide is present in dust obtained by grinding plant parts.

29. The method of claim 28, wherein said plant parts are selected from the group consisting of seeds and leaves.

30. The method of claim 28, where said dust is dispersed in water.

31. The method of claim 28, where said dust further comprises a Cry protein.

32. The method of claim 28, where said dust is combined with a Cry protein.

33. An isolated polypeptide that binds a Bacillus Cry protein, wherein said polypeptide comprises at least 75 amino acid residues, and said polypeptide is at least 85% identical with at least 75 contiguous amino acid residues of a receptor selected from the group consisting of SEQ ID NO:2 and SEQ ID NO:8.

34. A plant that produces the polypeptide of claim 33.

35. The plant of claim 34, wherein said polypeptide is produced in seeds.

36. The plant of claim 34, wherein said plant further produces a Cry protein.

37. The plant of claim 34, wherein said polypeptide is produced in leaves.

38. An isolated receptor comprising an amino acid sequence encoded by a polynucleotide that hybridizes under stringent conditions, of 0.1×SSPE at 42° C., with a nucleic sequence that encodes a protein selected from the group consisting of SEQ ID NO:2 and SEQ ID NO:8.

39. A method of inhibiting a mosquito, said method comprising providing a polypeptide to said mosquito for ingestion, wherein said polypeptide comprises a fragment of a midgut cadherin ectodomain from an insect, and said fragment binds a Bacillus Cry protein.

40. The method of claim 39, said method further comprising providing a Bacillus thuringiensis Cry4 protein to said mosquito for ingestion.

41. The method of claim 40, wherein said Cry4 protein is selected from the group consisting of a Cry4A protein and a Cry4B protein.

42. The method of claim 40, wherein said Cry protein is a Cry4Ba protein.

43. The method of claim 40, wherein said Cry protein is a Cry4BRA protein.

44. The method of claim 39, wherein said mosquito is of a genus selected from the group consisting of Anopheles, Aedes, and Culex.

45. The method of claim 39, where said cadherin is from a dipteran insect.

46. The method of claim 39, where said cadherin is from a mosquito.

47. The method of claim 46, where said cadherin is from a mosquito of a genus selected from the group consisting of Anopheles and Aedes.

48. The method of claim 39, where said cadherin is selected from the group consisting of AgCad or AgPCAP

49. The method of claim 39, where said polypeptide comprises an amino acid sequence selected from the group consisting of SEQ ID NO:4 and SEQ ID NO:10 (CR11-MPEDs)

50. The method of claim 39, where said polypeptide comprises SEQ ID NO:6 (CR9-11)

51. The method of claim 39, where said polypeptide comprises CR11.

52. The method of claim 39, where said cadherin is from a lepidopteran insect.

53. The method of claim 39, where said cadherin is BtR1.

54. The method of claim 39, where said polypeptide comprises an amino acid sequence selected from the group consisting of CR12-MPED (SEQ ID NO:14) and CR12.


SEQUENCE LISTING (37 sequences)

SEQ ID NO: 1 - DNA, 5208 bp, Anopheles gambiae
atgaaatgtgttgctagtaaatttaacatgtggttgcacttgggttggttgctggggttgctgctggtcctgttgccgttggtccgatgccaaggatggggcgaaccacggttcgagacgggaaatgtggaaaatatatccctcgccgcatacaacgaggcgcagcttcagcaagatgtctggatggtggaggagatggatgcaccgttcgtgctgctctacatcaattaccaaggaccgtccgagcctacgatacgcgagtcaccggccgatcttgacgcaaggctacagctgtccgagggtggccgctggtcgatcgtaatcaatcgccggcaggactacgaggtgcatcagcgtagcagtctcattctgctggccgtcgaatccacggctatcccgtacgcgatcgtggtcaacttggtgaacgtgctggacaatgcgcccgtcatgacggcccaaggtagctgtgagattgaggagttgcgcggggactttgtgacggactgtctgtttaacgtgtaccatgcggacgggttcgaggagaatggcattggcaattcgagcacgaacgagctgtcgttcgagatcggtgatgtggccggtgcgcgggaccactttacgtacgtgccctccacggtgaccccttcccagccgatctacaacaagctgttcaatttgaaagttttaaagcagctggactacaccgagaacgctatatttaacttcatcaccactgtgtacgacctagaccggacgcactctttcaagatgagtacgatcgttcaggtacgcaacgtcgatagccggcctccgatctttagccgaccgttcaccagcgaacgaatcatggaaaaggaaccattttacgcgaccgtgatcgcaatcgaccgagacactggactaaacaaaccgatctgttacgagctgacggctctagtcccggaatatcagaaatatttcgaaattggacaaactgatggaaagctgaccgtgcacccgattgatcgagatgcggaacagaacgagctgtacacctttacgattgtagcgtacaagtgtcacaaccggctgctgaacacctccagcgagggtgcaatcattttgctggacaaaaatgacaacattcccgaaatatacatgaagccgcttgagctggagttttgggaaaacaccatcatggagctgccattcgacgagctcgtcattcacgatcgcgatctcggcgagaatgcccggtacgaggtgcggctggccgaaacggtagcaggcgtacagcagacagcggactcgttcaccatcatccctggcaacgggtaccagcgtgtatcgttcacgatcaacatcaacaatgcgaccagcctggactatgagctgccggagcgtcaaacgtttgtgctgcacgttaccgctcacgaaccgatcgaaccgacgcacgaaagcacccagccgattacgattcggctcaaaaactggaacgatgaggtaccgaagtttggccgcgatgagtaccagatcagcgtgcccgaaacgatcggagcgggcgagctgttggcaacgatcaccgtgaccgatcgcgacatagacgatggtattaagctgtccgcactgggacggttggctgaaagtttaagcgtcaccgagctgccggtcagtgccgagcccgagactaacctccccctgtacgggttcgaaatcactaccaaggtgggagacatcttcgactacgatattgcgaaagaggtgatagtgcagctgcaggctgaagatacgctgcgcacggctaaacaagaaagcctgcatcagatcttctcgcagctaaccatcacggtgatagacgtgaacaataagccgccccaaattacgctgcctcgcggtacgatgcacattttggaaaactcggtcgccgacagtgcggttatcattggcgaggagcagatagcacagatcatcggcacagatcccgacaccgaggcggagctggagtttagtatcgattggagcaacagctacggcaccaagagcggaatccgggcgaaggcggaaacatacgaaaactgtttctacatacacgaggaaaaggtaaaccgtcagcgaacgatcggaaccatccgtgtgaatcccacgttccccctggacgtcgatcacgaaatgtacgacacactgttcctggtcattcggctggtggaccggaatcagacgatcctgcccaacacggtggaaactgtagtggcgatacagattgacgatgagaacgataatgctccttactttgacaacagcacgctcacggtggtacggtcggttaaggaacgatcggactcgggcgtaacgatcggtaacatcatcgctcacgacattgacggaccaggaaataacgaaataacgttcgcgatggaaccaatcgatccggcccacaaaggatggatgaacattgacgacaacggtaccgtaagggtggagggcaatcgatcgatcgactgtgacatcccgcccatcgacaaggtgctacagaacgtaaccatatcggactggaagtggagtaactggcacgtgtttgaaattgtcctgatggacaccaacaacaagcagccgtatcacgatcccttcccgaacgatgggcaggtgtaccagtttgagaaaataccctccaatacagccatcgtgcgggtggagggcaaagatcaggatcgcgatgtcccgtaccataccgtgtcgtacgagatcaactatcgggattttccgcagctgcagcgttacttcgaggtggacagtaccgggcgggcgtacgtgaaggaaaacaacgatctgctcgatcgagatgcgggtcttgagagcattatgattaacatcgtgatgctggataatgcgggcggatatgacattcaaaatcgtgtatcgacaaacatcaatctcactctgctcgacatcaacgatcatacgccaaagctaccggagctggcagcggacgaactgaaggtgtcggagaacgccaagcagggctacatcgtaaagacaccctttgctgcacttgacctagatgacaagcgtacgccaaatgcaaagatcaactactacatcgaagagatgacgccagagccggaaactcccctattctcgctagaaaatatagacgaatacaatgcggtgccaagggtggctcaagatctgaaaggattttacggaacgtggacactgaaaattaaggcctgtgatcgtggcagtgagtacgaaccaataattccactaacggaagaaccgaaggacaattgcgaaacacgcgactacgagctgacggtggaaccgttcaactacaacactcccagcattacctacccctcccgtagcgcacagctccgtctaaagtacgaatcgctacaaaacggacgcccgctggtcgagactaatgggtccccgttgccgaaattcgaagccatcgatgatgatggtggcatatatggagacgtaacgttttcactgaccagcacaaatgacggtgagcaggaccacgaagtgttccgggtggataaagttgacaacaaaacgggcctgctagtgttggaaaattcgctcgccgtacaaccgttcccgaaaaactacagcatcaccgtgattgccagggatggcggtgacaggcagtcggaagccgctattcacgtcgtcttcatcaacatgacgggcgagccggcctttctggagccgaccttcgatacggactttacagaaaatgaagagggtcgcgatgagagacggcagctaccgtttgccgaggatccgaagaacgcgggccttcccccgggcgctgagacgaacgtgtactatttcatcgataagacgtacggtaatgcgagccatctgtttcaactggaccgcgtcagcaatgtgctgcagctggcccagctgctcgaccgggaagagataccgacgcccgaaatccgaatcgtggccaccaacaacgaaaatagcccgccggatacggtgctcgaatcgtccccctcgctgctggtcgtccgcatcaaggtgaacgatgtgaacgacaatccgccggtgtttcagcagcggctgtacgccgccggcatcaccacgaacgaccgcgtcccgaaggcactgtttcgcgtgtacgccgaagatccggacgaggacgaaatcatccggtacgagttggtgaacgggacggcggtcggtgaaaacctgcaaacggacgggctgcccttccggctccatccggacagtggagagctaacgctgacgtccaaggtacagccgaaccagaacgggtactatcagctcaccctgatcgcgttcgatcgggacgatacgcacaatgatacggtgccggccaaggtgtacatcgtgtcggaatcgaaccgcgtgacgttcgtgtttctgaacagcgtcgaggagattgatcaaccggacgtgcggaagtttcttgcccaagagctgaccggtgcgtacgaaatggaatgcaacatcgacgacatcgaccagacgacggcgagcgacggtagacaggccggggggtctagcagcgccctcacggacgttcggacccacttcatccaggacaatcaggcggtggaggcgagccggatacagcagcgatcctcgaaccggacgttcgtcaccgtgctcaagacgacgctgcgcactcgcggcctctccctgcaggacgtaccgcccctggcgacggaagcgctgaccgaggcggacgaaacgctgcagatcatcctgattgtggtatcggcggccctagccgtcctgtgcgtgatactgttcgtggcgttcttcatcaagatccgcagcctaaatcgccagctgaaggccctgtccgcgaccgattttgggtcgatttcgtccgagctgaatgggaagccgacgcgcaacgtgcccacaacgaacatcttctcgatcgagggctccaatccggtgcttaacgataacgagttccgagaccggatgggtggtggtggtggcggtgtttatgacgatctcagtctacaatcggaagaatccgacttcaacgacgtggacagggacattttcgcaccgaagcgaaaggagagtcttaatcccgcgctcctggagcacatacgccagcgttcgctaaacccaatggcgaacggaaccgacaagagcaacgacggtgcgcctacctccaaccataaaaagctcgacgaaactgacgacgagctgtcccaccggttctag

SEQ ID NO: 2 - protein, 1735 aa, Anopheles gambiae
MKCVASKFNMWLHLGWLLGLLLVLLPLVRCQGWGEPRFETGNVENISLAAYNEAQLQQDVWMVEEMDAPFVLLYINYQGPSEPTIRESPADLDARLQLSEGGRWSIVINRRQDYEVHQRSSLILLAVESTAIPYAIVVNLVNVLDNAPVMTAQGSCEIEELRGDFVTDCLFNVYHADGFEENGIGNSSTNELSFEIGDVAGARDHFTYVPSTVTPSQPIYNKLFNLKVLKQLDYTENAIFNFITTVYDLDRTHSFKMSTIVQVRNVDSRPPIFSRPFTSERIMEKEPFYATVIAIDRDTGLNKPICYELTALVPEYQKYFEIGQTDGKLTVHPIDRDAEQNELYTFTIVAYKCHNRLLNTSSEGAIILLDKNDNIPEIYMKPLELEFWENTIMELPFDELVIHDRDLGENARYEVRLAETVAGVQQTADSFTIIPGNGYQRVSFTININNATSLDYELPERQTFVLHVTAHEPIEPTHESTQPITIRLKNWNDEVPKFGRDEYQISVPETIGAGELLATITVTDRDIDDGIKLSALGRLAESLSVTELPVSAEPETNLPLYGFEITTKVGDIFDYDIAKEVIVQLQAEDTLRTAKQESLHQIFSQLTITVIDVNNKPPQITLPRGTMHILENSVADSAVIIGEEQIAQIIGTDPDTEAELEFSIDWSNSYGTKSGIRAKAETYENCFYIHEEKVNRQRTIGTIRVNPTFPLDVDHEMYDTLFLVIRLVDRNQTILPNTVETVVAIQIDDENDNAPYFDNSTLTVVRSVKERSDSGVTIGNIIAHDIDGPGNNEITFAMEPIDPAHKGWMNIDDNGTVRVEGNRSIDCDIPPIDKVLQNVTISDWKWSNWHVFEIVLMDTNNKQPYHDPFPNDGQVYQFEKIPSNTAIVRVEGKDQDRDVPYHTVSYEINYRDFPQLQRYFEVDSTGRAYVKENNDLLDRDAGLESIMINIVMLDNAGGYDIQNRVSTNINLTLLDINDHTPKLPELAADELKVSENAKQGYIVKTPFAALDLDDKRTPNAKINYYIEEMTPEPETPLFSLENIDEYNAVPRVAQDLKGFYGTWTLKIKACDRGSEYEPIIPLTEEPKDNCETRDYELTVEPFNYNTPSITYPSRSAQLRLKYESLQNGRPLVETNGSPLPKFEAIDDDGGIYGDVTFSLTSTNDGEQDHEVFRVDKVDNKTGLLVLENSLAVQPFPKNYSITVIARDGGDRQSEAAIHVVFINMTGEPAFLEPTFDTDFTENEEGRDERRQLPFAEDPKNAGLPPGAETNVYYFIDKTYGNASHLFQLDRVSNVLQLAQLLDREEIPTPEIRIVATNNENSPPDTVLESSPSLLVVRIKVNDVNDNPPVFQQRLYAAGITTNDRVPKALFRVYAEDPDEDEIIRYELVNGTAVGENLQTDGLPFRLHPDSGELTLTSKVQPNQNGYYQLTLIAFDRDDTHNDTVPAKVYIVSESNRVTFVFLNSVEEIDQPDVRKFLAQELTGAYEMECNIDDIDQTTASDGRQAGGSSSALTDVRTHFIQDNQAVEASRIQQRSSNRTFVTVLKTTLRTRGLSLQDVPPLATEALTEADETLQIILIVVSAALAVLCVILFVAFFIKIRSLNRQLKALSATDFGSISSELNGKPTRNVPTTNIFSIEGSNPVLNDNEFRDRMGGGGGGVYDDLSLQSEESDFNDVDRDIFAPKRKESLNPALLEHIRQRSLNPMANGTDKSNDGAPTSNHKKLDETDDELSHRF

SEQ ID NO: 3 - DNA, 636 bp, Anopheles gambiae
ggcatcaccacgaacgaccgcgtcccgaaggcactgtttcgcgtgtacgccgaagatccggacgaggacgaaatcatccggtacgagttggtgaacgggacggcggtcggtgaaaacctgcaaacggacgggctgcccttccggctccatccggacagtggagagctaacgctgacgtccaaggtacagccgaaccagaacgggtactatcagctcaccctgatcgcgttcgatcgggacgatacgcacaatgatacggtgccggccaaggtgtacatcgtgtcggaatcgaaccgcgtgacgttcgtgtttctgaacagcgtcgaggagattgatcaaccggacgtgcggaagtttcttgcccaagagctgaccggtgcgtacgaaatggaatgcaacatcgacgacatcgaccagacgacggcgagcgacggtagacaggccggggggtctagcagcgccctcacggacgttcggacccacttcatccaggacaatcaggcggtggaggcgagccggatacagcagcgatcctcgaaccggacgttcgtcaccgtgctcaagacgacgctgcgcactcgcggcctctccctgcaggacgtaccgcccctggcgacggaagcgctgaccgaggcg

SEQ ID NO: 4 - protein, 212 aa, Anopheles gambiae
GITTNDRVPKALFRVYAEDPDEDEIIRYELVNGTAVGENLQTDGLPFRLHPDSGELTLTSKVQPNQNGYYQLTLIAFDRDDTHNDTVPAKVYIVSESNRVTFVFLNSVEEIDQPDVRKFLAQELTGAYEMECNIDDIDQTTASDGRQAGGSSSALTDVRTHFIQDNQAVEASRIQQRSSNRTFVTVLKTTLRTRGLSLQDVPPLATEALTEA

SEQ ID NO: 5 - DNA, 978 bp, Anopheles gambiae
gggtccccgttgccgaaattcgaagccatcgatgatgatggtggcatatatggagacgtaacgttttcactgaccagcacaaatgacggtgagcaggaccacgaagtgttccgggtggataaagttgacaacaaaacgggcctgctagtgttggaaaattcgctcgccgtacaaccgttcccgaaaaactacagcatcaccgtgattgccagggatggcggtgacaggcagtcggaagccgctattcacgtcgtcttcatcaacatgacgggcgagccggcctttctggagccgaccttcgatacggactttacagaaaatgaagagggtcgcgatgagagacggcagctaccgtttgccgaggatccgaagaacgcgggccttcccccgggcgctgagacgaacgtgtactatttcatcgataagacgtacggtaatgcgagccatctgtttcaactggaccgcgtcagcaatgtgctgcagctggcccagctgctcgaccgggaagagataccgacgcccgaaatccgaatcgtggccaccaacaacgaaaatagcccgccggatacggtgctcgaatcgtccccctcgctgctggtcgtccgcatcaaggtgaacgatgtgaacgacaatccgccggtgtttcagcagcggctgtacgccgccggcatcaccacgaacgaccgcgtcccgaaggcactgtttcgcgtgtacgccgaagatccggacgaggacgaaatcatccggtacgagttggtgaacgggacggcggtcggtgaaaacctgcaaacggacgggctgcccttccggctccatccggacagtggagagctaacgctgacgtccaaggtacagccgaaccagaacgggtactatcagctcaccctgatcgcgttcgatcgggacgatacgcacaatgatacggtgccggccaaggtgtacatcgtgtcggaatcgaaccgcgtgacgttcgtgttt

SEQ ID NO: 6 - protein, 326 aa, Anopheles gambiae
GSPLPKFEAIDDDGGIYGDVTFSLTSTNDGEQDHEVFRVDKVDNKTGLLVLENSLAVQPFPKNYSITVIARDGGDRQSEAAIHVVFINMTGEPAFLEPTFDTDFTENEEGRDERRQLPFAEDPKNAGLPPGAETNVYYFIDKTYGNASHLFQLDRVSNVLQLAQLLDREEIPTPEIRIVATNNENSPPDTVLESSPSLLVVRIKVNDVNDNPPVFQQRLYAAGITTNDRVPKALFRVYAEDPDEDEIIRYELVNGTAVGENLQTDGLPFRLHPDSGELTLTSKVQPNQNGYYQLTLIAFDRDDTHNDTVPAKVYIVSESNRVTFVF

SEQ ID NO: 7 - DNA, 5646 bp, Anopheles gambiae
atggaacagaatcgctcaaccgataagctacagatggaaatactcaagcgaaccgtctgccggctcaaaccgtcggcccaccggtgcctattggccggatctcattccttcacgcagctagtgctctgcctcatcctaagcgccaccctggtcagctgcaaccgtgcgccagtgtttctgatcgacgatcatgcggaaatagttatacgattgagggagttccccgagacgcccgtggggacgctgatctaccggctgcgtgggtacgatgcggatggcgatccgcttacctttggcgtgcagaagagtgccgacagccacatcataagactgaaacagaacacttccagcgaagcgttcgtctacctcaaccacgagctcgaccgggaggcacgcgaagagtacacgctcatcctcacgctcaccgatgggcggctcggtgagggtaacttcgtgacgcagagcttcctgctgctggtggaagatatcaacgataatgagccgatctttaaaccgttcgcctctgtgctggaggtagcggaggacagtccgccggggattttgaccacgctggaggccgtcgataaggacgagggtgcgtacgggcaggtcgtgtactacatccaagggttaagcgaggagaacaacgtgttttcgatctccacttccaatgggaagggtgtggtacggctggcccgagcgctggactacgagcggcaacatttctatcacatcaatgtgctggcagtggaccgggcgatacaggggaggatcaacactggaactgcagcactgctcgtgagagtgaaggacgtggaggaccagccgcccgagtttctggtaacgcaaccggtggtacgaatatccgaggatgctccaattggaacggaagtgattgcgaggatgatctattctctttcaacagtcaaagctgtggatggtgatcgaggaattaataatcgaattatttacgggatttcaaacaacggcagtgaactgtttgagattgatcggttgaagggttcactgcgaacgaaacagaagctcgacagggaggactccacgaatcccatcaacggtgctttcatactggaagtggttgcgattgaagagagcaagctacagcctgcgccttcctcaacaatggagatcaccgtgatcgtgacggatgtgaacgacgaaataccacgcttccgaagcgacggctatgagtgtgagattggcgagaacgcacaggagaacacgctggcccggttcatcgacggcagcatcaacgaggtgtttgactacgatcagggtaaaaatggcacgtttcgactgtcgctccatccgccgagtgacatcttcgaggtgattccaaagcgagcgatcaacgaggccacattcgggttgcgtgtgaaggatccgtccatgctcgattacgagcgggttcgggagctgtccctcacagtggtggcgagtgaggttgagtccgctggtcgtaccagtaccgcccagatacgggtggtcgtgctggatcagaacgataactttcccgagttttcacagcccgtctacgacattgatgtaccggagaatgtgatagccggtacggtgttgttgcagctacaagcaacggatagtgattcgggcctgttcggcacggagggtgtacggtacgcgaacctgactggtagtatctcgagctttctccatctggatccacacgctggcacggtgacacttatggcatcggagagtcccgtgtttgatcgtgaaatcatccaaaagcactacctctcggtggaggcccgtgataatggcggtcggggcaatcggaacacggtgccactgatactgaacgtgctcgatgtgaacgacaatgccccagtgtttgtgcagaagcggtacgaggtgagactgaaggagaatgcgtttgagtttgagtccccgatcgtggtggaagcgcgtgacagcgacctagaaggctcgccgaacagtgcggtagagtatcggttgattggtgctcaccattccgactacttccatgtggataggcgcacgggaagactctcagtgcgtgaagcagttgactttgaacggttggaaagtagtggcggtagtggtgacacgcgaaccattgcacttaccattgaggcggcggatggtggggagccaccgctgactgcccaggtcgaggtgacggtgtacgtgcaggacgtgaacgattatgcgcccgtttttctggagtcacagtacgcgatcgtcataccggaggacacgccgagcgggttgcccgtgctgcgcgtgaccgcgatggatggagatgggtcttttcccaacaaccacgtcacgtaccggatacagcagggtggtgatggtaggttcgtaataggagctagtacaggcgagatatcgatcacgcacggtgcatcgctggatccgaatctgctcgcgcccgatgcgctgggctcggggtcaacgtacgtgctggaggtgttcgctaccgatggtggcaatggagatcagcagctgcaaggatcctgtctggtgaacaatacgcccgtcggcaccgaagtgtaccggctgatggccaccgatccggacgagggtgcaatgctgcggtactacatcgatcgaagcctatcggagggcaagactgaggagggtgcactggtgaagctggacgattatgactttgcggcggccttcatactgaacgaaacgaatggactgctcaagatagcaaagctgctggatcgcgaaaagattgccgaaatcaagctggcctgtgtggtggaggacgtagcggccgagcggggtgaccagatggcaaacaccttcctcaagatcaccgtgctcgacgagaacgacaacaatcccaagttccgcaaaccgttctacaaacactccattgcggaaaacagtcagtacggtgtggcggtatgtacggtggtggccgaagatgccgatcagaacaaaacggtcaagtacagcttggaaggggagaagggtgtgctggagttgctgcacgttgatgacgagacgggggagattgtggtgcgcaatcggatcgatcatgaggagtacagttggttgaacttctcggtacgggcggctgatacgggcacgccgccgagagcgtcctttgtggaggtgttcgtgcaggtgctggatgagaacgacaacaatccttactttgtggacagcgtgaacgattactacgtgtcggagaatgcaagcgtgggagcggagatagcgatcatactggcgaaggatctagattcgggggatttcggacgcatcacgtacatactggatcgtgttagctcgaaggagaagtttagcattgatccggagaaaggaatactacgagtagcgggagcattggatcgggaagagacagctgagtatatgttggcagtagaggcatgggataactatccttacggctatctcaacggtgaaagccgcaacgcttttaaacacatactgatacacgtgctggacgacaatgacaacgttcccgtgatccagaaaccttccgggtgtagtatgatcaccgagtaccacaacatcaacgatccgatcgtgaagctgcgggcaaccgatgcggacgacccaaccaatggaaacggtcaactgagcttcgacattgtggacccctcggggatattctacatccagcaggtgtcggcccagtacgcagaaatatattcccgcgggccgctaaagaatctgcacggcaactacacgctggagctgatagtgagcgatcttggcggcgtaccgaacacggcccgagagtcggtggacatctgtgtgacggatttcaacgaccatgcgccagtgtttgttgtccccagtggaaatacaactgtgaaggtgtttgagaacacaacgctcggtaagccgttctttcaagtgcacgcgtacgatgaagatgtcggcgaaaatgcgattgtgcgctaccgactgaagatggacacgatgggcaactttcgcaagttttccctcgacaaggagacgggcgagcttagcctcgctgcgcctctcgatcgcgagcagcagatgatgtacgatctgcgcatcgaagcgtacgatcagggtataccgacgccgctcagcagtacggtcgatctgatcgtgtacgtgcgggacgtcaacgacaatctacctcagtttttgctcaaggaaatcagtcttaactttacggagcacatgacaccgggcacggaaagaatacgcctgcccgacacggtcgaccaggactatctggacccgctggacggtccggcggcgagtgtggtttgctactacatcgtgcacggcaacgaggatggacactttggactggatccagtgtcacacgatctaacggtggagaaagagctcgatcgggagaaaaaatctctgtacaagctgcacattaaggcgacggaggagtgcacgaacgcgaatctatcgttggacacgaccagtcactcgggcaatctcctaaaggctactgtgtacataaacgacatcaacgacaactcgccagtgtttgagtcgaaaatattcaccggcggtatctcaacgtcgtcgctgttcggtgccacaattcttcagctgcaggccacggacgaagacgatggattgaacggattggttcggtactatcggcacggcgaggtgcggaaaacgcttgccgaggggttggacgatttgcgcagcgatccgttcctggtcgaggcggacacgggccaagtgttgctgaactttttcccccagaagtcgatgcgcggctactttgacttttccgtgctggcgaacgattcgtacggctgccatgatcgggcgcacgtgttcatctatctgatacgggaggatcagcgggtaaagtttatactgcgccaacggccgtccgagattcgtcacaacattcaaagttttcgcgagatcttgagcaatgtgagtggatgcatcgtgaacattgacgatatacgggtgcacgagaatccggacggttcggttgataggaccaagagcgacatgtttatgcatctggtcgatcagaagaacaactccgtgctggaggtacatcaggtgctgcagttgctggatacgcacgtggaaaagcttgatcgactgtttaaggaattcaacgtcttggacactcaagcgtcggagctggtccaaacggcagagatggacgagctgtccgtcaacataatatggctgtttgtgaccaacattttgctcggcgcgctgctgatcgttgtaataggactgtcgatctcacagcgtttatcctaccgcagacagctacgggcagcgaagatagctgcctttggctccacgggtcctagtcgcatgtatcaggaagtgctcggcgccgtaccgaacacgaacaagcacagcatgaagggcagcaacccgatctggatcggcagcggcacaccggagggcgaatgggcgaaggacgagttcgacaagtgcaaggatgcgatcgacgcacagtacgaacgatcgctcagctcgggcttcttcatcgacaactgtctgcagtacgaggcacggaagggtttcgccggggccgaagcgaacaacgccgccaactatcagctcaagcgggacagcgagacgacgctgtgcgcccggaacttggaaacgaccgaactgtaa

SEQ ID NO: 8 - protein, 1881 aa, Anopheles gambiae
MEQNRSTDKLQMEILKRTVCRLKPSAHRCLLAGSHSFTQLVLCLILSATLVSCNRAPVFLIDDHAEIVIRLREFPETPVGTLIYRLRGYDADGDPLTFGVQKSADSHIIRLKQNTSSEAFVYLNHELDREAREEYTLILTLTDGRLGEGNFVTQSFLLLVEDINDNEPIFKPFASVLEVAEDSPPGILTTLEAVDKDEGAYGQVVYYIQGLSEENNVFSISTSNGKGVVRLARALDYERQHFYHINVLAVDRAIQGRINTGTAALLVRVKDVEDQPPEFLVTQPVVRISEDAPIGTEVIARMIYSLSTVKAVDGDRGINNRIIYGISNNGSELFEIDRLKGSLRTKQKLDREDSTNPINGAFILEVVAIEESKLQPAPSSTMEITVIVTDVNDEIPRFRSDGYECEIGENAQENTLARFIDGSINEVFDYDQGKNGTFRLSLHPPSDIFEVIPKRAINEATFGLRVKDPSMLDYERVRELSLTVVASEVESAGRTSTAQIRVVVLDQNDNFPEFSQPVYDIDVPENVIAGTVLLQLQATDSDSGLFGTEGVRYANLTGSISSFLHLDPHAGTVTLMASESPVFDREIIQKHYLSVEARDNGGRGNRNTVPLILNVLDVNDNAPVFVQKRYEVRLKENAFEFESPIVVEARDSDLEGSPNSAVEYRLIGAHHSDYFHVDRRTGRLSVREAVDFERLESSGGSGDTRTIALTIEAADGGEPPLTAQVEVTVYVQDVNDYAPVFLESQYAIVIPEDTPSGLPVLRVTAMDGDGSFPNNHVTYRIQQGGDGRFVIGASTGEISITHGASLDPNLLAPDALGSGSTYVLEVFATDGGNGDQQLQGSCLVNNTPVGTEVYRLMATDPDEGAMLRYYIDRSLSEGKTEEGALVKLDDYDFAAAFILNETNGLLKIAKLLDREKIAEIKLACVVEDVAAERGDQMANTFLKITVLDENDNNPKFRKPFYKHSIAENSQYGVAVCTVVAEDADQNKTVKYSLEGEKGVLELLHVDDETGEIVVRNRIDHEEYSWLNFSVRAADTGTPPRASFVEVFVQVLDENDNNPYFVDSVNDYYVSENASVGAEIAIILAKDLDSGDFGRITYILDRVSSKEKFSIDPEKGILRVAGALDREETAEYMLAVEAWDNYPYGYLNGESRNAFKHILIHVLDDNDNVPVIQKPSGCSMITEYHNINDPIVKLRATDADDPTNGNGQLSFDIVDPSGIFYIQQVSAQYAEIYSRGPLKNLHGNYTLELIVSDLGGVPNTARESVDICVTDFNDHAPVFVVPSGNTTVKVFENTTLGKPFFQVHAYDEDVGENAIVRYRLKMDTMGNFRKFSLDKETGELSLAAPLDREQQMMYDLRIEAYDQGIPTPLSSTVDLIVYVRDVNDNLPQFLLKEISLNFTEHMTPGTERIRLPDTVDQDYLDPLDGPAASVVCYYIVHGNEDGHFGLDPVSHDLTVEKELDREKKSLYKLHIKATEECTNANLSLDTTSHSGNLLKATVYINDINDNSPVFESKIFTGGISTSSLFGATILQLQATDEDDGLNGLVRYYRHGEVRKTLAEGLDDLRSDPFLVEADTGQVLLNFFPQKSMRGYFDFSVLANDSYGCHDRAHVFIYLIREDQRVKFILRQRPSEIRHNIQSFREILSNVSGCIVNIDDIRVHENPDGSVDRTKSDMFMHLVDQKNNSVLEVHQVLQLLDTHVEKLDRLFKEFNVLDTQASELVQTAEMDELSVNIIWLFVTNILLGALLIVVIGLSISQRLSYRRQLRAAKIAAFGSTGPSRMYQEVLGAVPNTNKHSMKGSNPIWIGSGTPEGEWAKDEFDKCKDAIDAQYERSLSSGFFIDNCLQYEARKGFAGAEANNAANYQLKRDSETTLCARNLETTEL

SEQ ID NO: 9 - DNA, 639 bp, Anopheles gambiae
ggtatctcaacgtcgtcgctgttcggtgccacaattcttcagctgcaggccacggacgaagacgatggattgaacggattggttcggtactatcggcacggcgaggtgcggaaaacgcttgccgaggggttggacgatttgcgcagcgatccgttcctggtcgaggcggacacgggccaagtgttgctgaactttttcccccagaagtcgatgcgcggctactttgacttttccgtgctggcgaacgattcgtacggctgccatgatcgggcgcacgtgttcatctatctgatacgggaggatcagcgggtaaagtttatactgcgccaacggccgtccgagattcgtcacaacattcaaagttttcgcgagatcttgagcaatgtgagtggatgcatcgtgaacattgacgatatacgggtgcacgagaatccggacggttcggttgataggaccaagagcgacatgtttatgcatctggtcgatcagaagaacaactccgtgctggaggtacatcaggtgctgcagttgctggatacgcacgtggaaaagcttgatcgactgtttaaggaattcaacgtcttggacactcaagcgtcggagctggtccaaacggcagagatggacgagctgtccgtc

SEQ ID NO: 10 - protein, 213 aa, Anopheles gambiae
GISTSSLFGATILQLQATDEDDGLNGLVRYYRHGEVRKTLAEGLDDLRSDPFLVEADTGQVLLNFFPQKSMRGYFDFSVLANDSYGCHDRAHVFIYLIREDQRVKFILRQRPSEIRHNIQSFREILSNVSGCIVNIDDIRVHENPDGSVDRTKSDMFMHLVDQKNNSVLEVHQVLQLLDTHVEKLDRLFKEFNVLDTQASELVQTAEMDELSV

SEQ ID NO: 11 - DNA, 5154 bp, Manduca sexta
atggcagttgacgtccgaatcgctgccttcctgctggtgtttatagcgcctgcagttttagctcaagagagatgcgggtatatgaccgccatcccaaggctaccacgaccggataatttgccagtactaaactttgaaggccagacatggagtcagaggcccctgctccccgccccggagcgggatgacctgtgcatggacgcctaccacgtgataacagccaacctcggcacgcaggtcatctacatggatgaagagatagaagacgaaatcaccatcgccatacttaattataacggaccatcaactccgttcattgaactgccatttttatccggttcgtacaatctgctgatgccggtcatcaggagagttgacaacggggagtggcatctcatcatcacgcaaagacaggattacgagttgcccggcatgcagcagtacatgttcaatgtgcgcgtggacggccagtcgctggtggcaggcgtgtctctcgctatcgtcaacatagatgacaacgcgcccatcatacaaaacttcgagccttgccgggttcctgaactgggcgagccagggttgacagaatgcacataccaagtatcggacgcggacggacggatcagtacagagttcatgacgttcaggatcgacagcgttcgtggcgacgaggagaccttctacatcgaacggacgaatatccccaaccaatggatgtggctaaatatgaccttaggcgttaatacctcgctcaacttcgtcaccagtccgctgcatatattcagcgtgacagccctggactcgctcccgaacacccacacggtgactatgatggtgcaagtggcgaatgtgaacagccgtccgccgcgctggctggagatcttcgctgtccaacagtttgaagagaaatcttaccaaaacttcacagtgagggcgatcgacggagacactgagatcaatatgcctatcaactacaggctgatcacaaatgaggaagacacattcttcagcatcgaggccctgcctggtggaaaaagcggggctatattccacgtgtcaccaattgaccgcgacacactgcaacgagaggtgtttccacttacgatcgtcgcttacaaatacgatgaggaagctttctccacatcaacaaacgtggtcatcattgtgacagacatcaacgaccaaagacctgaacctatacacaaggaatatcgactggcgatcatggaggagacgcccatgaccctcaacttcgataaagaatttggatttcatgataaggatttaggtcaaaacgctcagtacacggtgcgtctagagagcgtggaccctccaggcgctgctgaggcattctacatagcgcctgaagtcggctaccagcgacagaccttcatcatgggcaccctcaatcactccatgctggattacgaagtgccagagtttcagagtattacgattcgggtggtagcgaccgacaacaacgacacgaggcacgtgggcgtcgctttggttcacattgacctcatcaattggaacgatgagcagccgatcttcgaacacgccgtgcagaccgtgaccttcgacgagactgaaggcgaggggttcttcgtcgccaaggcggttgcacacgacagagacatcggggatgtcgtcgagcatactttactgggtaacgctgttaacttcctgaccatcgacaaactcaccggcgacatccgcgtctcagctaacgactccttcaactaccatcgagaaagtgaattatttgtgcaggtgcgagctacagacacgctgggcgaacccttccacacgacgacgtcacagctggtcatacgactaaatgacatcaacaacacgccacccaccttacggctgcctcgaggcagtccccaagtggaggagaacgtgcctgatggccacgtcatcacccaggagttacgcgccaccgaccccgacaccacggccgatctgcgcttcgagataaactgggacacctctttcgccaccaagcaaggccgccaggctaaccccgacgagtttaggaattgcgtggaaatcgagaccatcttccccgagattaacaaccggggactggctatcggccgcgttgtagcgcgcgagatcagacacaacgtgaccatagactacgaggagtttgaggtcctctccctcacagtgagggtgcgggacctgaacaccgtctacggagacgactacgacgaatcgatgctcacaataactataatcgacatgaacgacaacgcgccggtgtgggtggaggggactctggagcagaacttccgcgtgcgcgagatgtccgcgggcgggctcgtggtgggctccgtgcgcgcggacgacatcgacggaccgctctacaaccaagtgcgatacaccattttccctcgtgaagacacagataaggacctgataatgatcgacttcctcacgggtcaaatttccgtgaacacaagcggcgccatcgacgcggacacccctcctcgcttccacctctactacacagtggtcgctagtgaccgatgctcgacagaagatcctgcagattgcccccctgatccgacttattgggaaaccgaaggaaatatcacaatccacatcaccgacacgaacaacaaggtcccgcaggcggaaacgactaagttcgataccgtcgtgtatatttacgagaacgcaacccacttggacgaggtggtcactctgatagccagtgatcttgacagagacgaaatataccacacggtgagctacgtcatcaattacgcagtgaaccctcgactgatgaacttcttctccgtgaaccgggagaccggcctggtgtacgtagactatgagacccaggggagtggcgaggtgctggaccgtgatggtgatgaaccaacgcaccgtatcttcttcaacctcatcgacaacttcatgggggaaggagaaggtaacagaaatcagaacgacacagaagttctcgttatcttgttggatgtgaatgacaatgctcctgaattgccaccgccgagcgaactctcttggactatatctgagaaccttaagcagggcgtccgtcttgaaccgcatatcttcgccccggaccgcgacgagcccgacacggacaactccagggtcggttacgagatcctgaacctcagcacggagcgggacatcgaagtgccggagctgtttgtaatgatacagatcgcgaacgtcacgggagagctggagaccgccatggacctcaagggatattgggggacgtacgctatacatatacgggcattcgaccacggcattccgcaaatgtccatgaacgagacatatgagctgattatccatccgttcaactactacgcgcctgagttcgtcttcccgaccaacgatgccgtcatacgacttgcgagggaacgagctgtaatcaatggagttctagcgacagtgaacggagagttcttggagcggatatcggcgactgacccggacggactccacgcgggcgtcgtcaccttccaagtggtaggcgatgaggaatcacaacggtactttcaagtagttaacgatggcgcgaacctcggctcgttgaggttactgcaagccgttccagaggagatcagggagttccggataacgattcgcgctacagaccagggaacggacccaggaccgctgtccacggacatgacgttcagagttgtttttgtgcccacgcaaggagaacctagattcgcgtcctcagaacatgctgtcgctttcatagaaaagagtgccggcatggaagagtctcaccaacttcctctagcacaagacatcaagaaccatctctgtgaagacgactgtcacagcatttactatcgtattatcgatggcaacagcgagggtcatttcggcctggatcctgttcgcaacaggttgttcctgaagaaagagctgataagagaacaaagtgcctcccacactctgcaagtggcggctagtaactcgcccgatggtggcattccacttcctgcttccatccttactgtcactgttaccgtgagggaggcagaccctcgtccagtgtttatgagggaattgtacaccgcagggatatccacagcggactccatcggcagagagctgctcagattacatgcgacccagtctgaaggcgcggccattacttatgctatagactacgatacaatggtagtggaccccagcctggaggcagtgagacagtcggctttcgtactgaacgctcaaaccggagtgctgacgcttaatatccagcccacggccacgatgcatggactgttcaaattcgaagtcacagctactgacacggccggcgctcaggaccgcaccgacgtcaccgtgtacgtggtatcctcgcagaaccgcgtctacttcgtgttcgtcaacacgctgcaacaggtcgaagacaacagagactttatcgcggacaccttcagcgctgggttcaacatgacctgcaacatcgaccaagtggtgcccgccaacgaccccgtcaccggcgtggcgctggagcacagcacgcagatgcgcggccacttcatacgggacaacgtacccgtactcgctgatgagatagaacagatccgtagtgacctagtcctcctgagctcgatacaaacaacgctggcggcgcgatcgctggtgttgcaggacttgttgaccaactccagcccggactcggcgcctgactcgagcctcacggtgtacgtgctggcctcactgtctgctgtgctcggtttcatgtgccttgtgctactgcttaccttcatcatcaggactagagcgctaaaccgacggttggaagccctgtcgatgacgaagtacggctcactggactctggattgaaccgcgccggcatcgccgcccccggcaccaacaaacacactgtggaaggctccaaccctatcttcaatgaagcaataaagacgccagatttagatgccattagcgagggttccaacgactctgatctgatcggcatcgaagatcttccgcactttggcaacgtcttcatggatcctgaggtgaacgaaaaggcaaatggttatcccgaagtcgcaaaccacaacaacaacttcgctttcaacccgactcccttctcgcctgagttcgttaacggacagttcagaaagatctag

SEQ ID NO: 12 - protein, 1717 aa, Manduca sexta
MAVDVRIAAFLLVFIAPAVLAQERCGYMTAIPRLPRPDNLPVLNFEGQTWSQRPLLPAPERDDLCMDAYHVITANLGTQVIYMDEEIEDEITIAILNYNGPSTPFIELPFLSGSYNLLMPVIRRVDNGEWHLIITQRQDYELPGMQQYMFNVRVDGQSLVAGVSLAIVNIDDNAPIIQNFEPCRVPELGEPGLTECTYQVSDADGRISTEFMTFRIDSVRGDEETFYIERTNIPNQWMWLNMTLGVNTSLNFVTSPLHIFSVTALDSLPNTHTVTMMVQVANVNSRPPRWLEIFAVQQFEEKSYQNFTVRAIDGDTEINMPINYRLITNEEDTFFSIEALPGGKSGAIFHVSPIDRDTLQREVFPLTIVAYKYDEEAFSTSTNVVIIVTDINDQRPEPIHKEYRLAIMEETPMTLNFDKEFGFHDKDLGQNAQYTVRLESVDPPGAAEAFYIAPEVGYQRQTFIMGTLNHSMLDYEVPEFQSITIRVVATDNNDTRHVGVALVHIDLINWNDEQPIFEHAVQTVTFDETEGEGFFVAKAVAHDRDIGDVVEHTLLGNAVNFLTIDKLTGDIRVSANDSFNYHRESELFVQVRATDTLGEPFHTTTSQLVIRLNDINNTPPTLRLPRGSPQVEENVPDGHVITQELRATDPDTTADLRFEINWDTSFATKQGRQANPDEFRNCVEIETIFPEINNRGLAIGRVVAREIRHNVTIDYEEFEVLSLTVRVRDLNTVYGDDYDESMLTITIIDMNDNAPVWVEGTLEQNFRVREMSAGGLVVGSVRADDIDGPLYNQVRYTIFPREDTDKDLIMIDFLTGQISVNTSGAIDADTPPRFHLYYTVVASDRCSTEDPADCPPDPTYWETEGNITIHITDTNNKVPQAETTKFDTVVYIYENATHLDEVVTLIASDLDRDEIYHTVSYVINYAVNPRLMNFFSVNRETGLVYVDYETQGSGEVLDRDGDEPTHRIFFNLIDNFMGEGEGNRNQNDTEVLVILLDVNDNAPELPPPSELSWTISENLKQGVRLEPHIFAPDRDEPDTDNSRVGYEILNLSTERDIEVPELFVMIQIANVTGELETAMDLKGYWGTYAIHIRAFDHGIPQMSMNETYELIIHPFNYYAPEFVFPTNDAVIRLARERAVINGVLATVNGEFLERISATDPDGLHAGVVTFQVVGDEESQRYFQVVNDGANLGSLRLLQAVPEEIREFRITIRATDQGTDPGPLSTDMTFRVVFVPTQGEPRFASSEHAVAFIEKSAGMEESHQLPLAQDIKNHLCEDDCHSIYYRIIDGNSEGHFGLDPVRNRLFLKKELIREQSASHTLQVAASNSPDGGIPLPASILTVTVTVREADPRPVFMRELYTAGISTADSIGRELLRLHATQSEGAAITYAIDYDTMVVDPSLEAVRQSAFVLNAQTGVLTLNIQPTATMHGLFKFEVTATDTAGAQDRTDVTVYVVSSQNRVYFVFVNTLQQVEDNRDFIADTFSAGFNMTCNIDQVVPANDPVTGVALEHSTQMRGHFIRDNVPVLADEIEQIRSDLVLLSSIQTTLAARSLVLQDLLTNSSPDSAPDSSLTVYVLASLSAVLGFMCLVLLLTFIIRTRALNRRLEALSMTKYGSLDSGLNRAGIAAPGTNKHTVEGSNPIFNEAIKTPDLDAISEGSNDSDLIGIEDLPHFGNVFMDPEVNEKANGYPEVANHNNNFAFNPTPFSPEFVNGQFRKI

SEQ ID NO: 13 - DNA, 618 bp, Manduca sexta
gggatatccacagcggactccatcggcagagagctgctcagattacatgcgacccagtctgaaggcgcggccattacttatgctatagactacgatacaatggtagtggaccccagcctggaggcagtgagacagtcggctttcgtactgaacgctcaaaccggagtgctgacgcttaatatccagcccacggccacgatgcatggactgttcaaattcgaagtcacagctactgacacggccggcgctcaggaccgcaccgacgtcaccgtgtacgtggtatcctcgcagaaccgcgtctacttcgtgttcgtcaacacgctgcaacaggtcgaagacaacagagactttatcgcggacaccttcagcgctgggttcaacatgacctgcaacatcgaccaagtggtgcccgccaacgaccccgtcaccggcgtggcgctggagcacagcacgcagatgcgcggccacttcatacgggacaacgtacccgtactcgctgatgagatagaacagatccgtagtgacctagtcctcctgagctcgatacaaacaacgctggcggcgcgatcgctggtgttgcaggacttgttgaccaactccagcccggactcggcgcct

SEQ ID NO: 14 - protein, 206 aa, Manduca sexta
GISTADSIGRELLRLHATQSEGAAITYAIDYDTMVVDPSLEAVRQSAFVLNAQTGVLTLNIQPTATMHGLFKFEVTATDTAGAQDRTDVTVYVVSSQNRVYFVFVNTLQQVEDNRDFIADTFSAGFNMTCNIDQVVPANDPVTGVALEHSTQMRGHFIRDNVPVLADEIEQIRSDLVLLSSIQTTLAARSLVLQDLLTNSSPDSAP

SEQ ID NO: 15 - protein, 8 aa, Heliothis virescens
GVLTLNFQ

SEQ ID NO: 16 - protein, 8 aa, Manduca sexta
GVLTLNIQ

SEQ ID NO: 17 - protein, 10 aa, Anopheles gambiae
GELTLTSKVQ

SEQ ID NO: 18 - DNA, 32 bp, Anopheles gambiae
ggtggccgctggtcgatcgtaatcaatcgccg

SEQ ID NO: 19 - DNA, 33 bp, Anopheles gambiae
cttaattttcagtgtccacgttccgtaaaatcc

SEQ ID NO: 20 - DNA, 33 bp, Anopheles gambiae
cgtgtatcgttcacgatcaacatcaacaatgcg

SEQ ID NO: 21 - DNA, 29 bp, Anopheles gambiae
atcatcgctcacgacattgacggaccagg

SEQ ID NO: 22 - DNA, 32 bp, Anopheles gambiae
gcaccctcgctggaggtgttcagcagccggtt

SEQ ID NO: 23 - DNA, 32 bp, Anopheles gambiae
ggtcccgcgcaccggccacatcaccgatctcg

SEQ ID NO: 24 - DNA, 33 bp, Anopheles gambiae
gttacctagtgtaccggctgctggcggccttaa

SEQ ID NO: 25 - DNA, 31 bp, Anopheles gambiae
cgttcgtctcagcgcccgggggaaggcccgc

SEQ ID NO: 26 - DNA, 30 bp, Anopheles gambiae
ccgtttgccgaggatccgaagaacgcgggc

SEQ ID NO: 27 - DNA, 36 bp, Anopheles gambiae
gaattcgcggccgcgggaattttttttttttttttt

SEQ ID NO: 28 - DNA, 35 bp, Anopheles gambiae
gacccatatggacgaaacgctgcagatcatcctga

SEQ ID NO: 29 - DNA, 35 bp, Anopheles gambiae
acacctcgaggaaccggtgggacagctcgtcgtca

SEQ ID NO: 30 - DNA, 21 bp, Anopheles gambiae
ggtatctcaacgtcgtcgctg

SEQ ID NO: 31 - DNA, 20 bp, Anopheles gambiae
cctccagcacggagttgttc

SEQ ID NO: 32 - DNA, 30 bp, Anopheles gambiae
cgagcatatggggtccccgttgccgaaatt

SEQ ID NO: 33 - DNA, 32 bp, Anopheles gambiae
cgctctcgagaaacacgaacgtcacgcggttc

SEQ ID NO: 34 - DNA, 35 bp, Anopheles gambiae
ttcaccatgggtatctcaacgtcgtcgctgttcgg

SEQ ID NO: 35 - DNA, 35 bp, Anopheles gambiae
catactcgagtgacggacagctcgtccatctctgc

SEQ ID NO: 36 - DNA, 35 bp, Manduca sexta
gtaccatatggggatatccacagcggactccatcg

SEQ ID NO: 37 - DNA, 35 bp, Manduca sexta
ggctctcgagaggcgccgagtccgggctggagttg